(19) United States
(12) Patent Application Publication
(10) Pub. No.: US 2009/0263811 A1
(43) Pub. Date: Oct. 22, 2009

(54) TARGET BASE DISCRIMINATION METHOD

(75) Inventor: Masanobu KIYOHARA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/412,812

(22) Filed: Mar. 27, 2009

(30) Foreign Application Priority Data
Apr. 2, 2008 (JP) 2008-096165

(51) Int. Cl. C12Q 1/68; C07H 21/04

(52) U.S. Cl. 435/6; 536/24.33

(57) ABSTRACT

A target base-specific primer for use in discrimination of the base type of a target base in a nucleic acid in a nucleic acid sample, which is capable of discriminating the target base clearly with less possibility of false positive, and a target base discrimination method using the target base-specific primer, are provided. The target base discrimination method of the present invention uses a target base-specific primer for use in discrimination of the base type of a target base in a nucleic acid having a target nucleotide sequence including the target base in a nucleic acid sample, in the case where a plurality of base types can be enumerated for the target base, wherein the primer has a target base-corresponding base which is a base complementary to a base predicted as the target base, and the base type of a mismatch base is a specific base type when the mismatch base refers to a base adjacent to the 5′ side of the target base-corresponding base, and a mismatch-corresponding base refers to a base adjacent to the 3′ side of the target base in the target nucleotide sequence.

### TECHNICAL FIELD

The present invention relates to a target base-specific primer for use in discrimination of the base type of a target base in a nucleic acid having a target nucleotide sequence including the target base in a nucleic acid sample, in the case where a plurality of base types can be enumerated for the target base, and a target base discrimination method using the target base-specific primer.

Priority is claimed on Japanese Patent Application No. 2008-096165, filed Apr. 2, 2008, the content of which is incorporated herein by reference.

### BACKGROUND ART

Gene polymorphism is considered to be a major factor contributing to the individual difference in the vulnerability against a specific disease such as cancer, the drug metabolizing capacity, and so forth. In particular, SNP (Single Nucleotide Polymorphism) is the most frequent polymorphism among gene polymorphisms, and is believed to appear in human genomes with an incidence of about 0.1%. As a matter of fact, the presence of SNP of as many as over three million has been hitherto elucidated in human genomes, suggesting that SNP may be very useful as a marker for genetic tests. Investigations on the relationship between SNP and diseases carried out so far have revealed a close association between SNP and drug sensitivity prediction or diseases such as diabetes and hypertension.

Known methods for discriminating SNP, that is, methods for discriminating the base type, among respective base types of SNP, in a nucleic acid in a nucleic acid sample include techniques in which a primer extension reaction with an allele-specific primer is utilized. Here, the term allele-specific primer refers to a primer which has a base complementary to a specific base type serving as the detection subject, among respective base types of the target SNP, and which yields a remarkable difference in the primer extension reaction efficiency depending on the base type of the target SNP of the template nucleic acid. Specifically, as for the target SNP, the primer extension reaction efficiency is increased if an allele having the base type of the detection subject is used as a template, while the primer extension reaction efficiency is lowered if an allele having another base type is used as a template. Accordingly, the base type of the target SNP can be specified, for example, by carrying out a PCR (Polymerase Chain Reaction) with a nucleic acid in the nucleic acid sample as a template, and an allele-specific primer, and analyzing the amount of the resultant PCR product.

That is, such SNP discrimination methods using an allele-specific primer are capable of readily discriminating SNP by analyzing the amount of the extension product resulting from a primer extension reaction, and thus are very useful in terms of cost, reaction time, convenience of operation, and the like, as compared to other SNP discrimination methods. For example, even if a simple oligonucleotide without any special modification is used as an allele specific primer, the amount of the extension product can be readily analyzed by using a general electrophoretic method. The method of analysis is not specifically limited to the electrophoretic method, and can also be achieved by performing a solid phase reaction and detection through analysis of quartz crystal microbalance (QCM) or surface plasmon resonance (SPR) phenomenon. More recently, methods of detecting PPi (pyrophosphate) that is a byproduct of a primer extension reaction, with use of a luciferase reaction have also been developed. Accordingly, approaches to simplification and acceleration of the SNP discrimination method with an allele-specific primer have been elaborately attempted all over the world. In particular, the sequence design of an allele-specific primer is a very important factor for its ability to discriminate SNP, and attempts have been made to develop an excellent allele-specific primer having higher SNP discrimination ability.

Several novel allele-specific primers have been so far developed. First, there has been proposed a primer having a base which corresponds to a polymorphic base site at any position in the primer (for example, see Patent Document 1). Here, the term “base which corresponds to a polymorphic base site” refers to a base in the primer which interacts with the polymorphic base site upon hybridization between the primer and a nucleic acid having the polymorphic base site. Thereafter, there has come to light a primer in which the polymorphic base site is limited within the 3′ end of the primer, that is, a primer which has a base complementary to any one of predicted base types of the target SNP, at the 3′ end (for example, see Patent Document 2). Such a primer in which the base at the 3′ end corresponds to the target SNP base and the other sequence is completely complementary to the target nucleotide sequence is the most typical allele-specific primer at present.

However, these allele-specific primers of Patent Documents 1 and 2 are insufficient in the SNP discrimination ability, and hence a problem of false positive occurs since the primer extension reaction efficiency is increased even with a nucleic acid having a base type different from the base type of the detection subject of the allele-specific primer as a template, unless the reaction conditions including the reaction time and temperature for the primer extension reaction, or the concentration of dNTPs to be used for the reaction, or the number of cycles if PCR is used, are strictly set.

In order to solve such a false positive problem, novel allele-specific primers have been developed in which the primer of Patent Document 2 is modified by artificially introducing a mismatch.

Examples of such primers include (1) an allele-specific primer in which the base at the 3′ end corresponds to the target SNP base and the second base from the 3′ end is introduced with a mismatch (for example, see Patent Document 3), and (2) an allele-specific primer in which the base at the 3′ end corresponds to the target SNP base and the second and third bases from the 3′ end are introduced with a mismatch comprising a combination of specific base types (for example, see Patent Document 4). In particular, regarding the allele-specific primer of Patent Document 4, all two-base combinations were investigated to select a combination of base types which particularly show high specificity, and such a base type combination is introduced as a mismatch.

In addition, examples of the primer in which the base corresponding to the target SNP base is not limited to the 3′ end of the primer, include (3) an allele-specific primer in which a base adjacent to the base corresponding to the target SNP base is substituted by a substituent which has no interactive action with a DNA-type base or an RNA-type base to thereby introduce a mismatch (for example, see Patent Document 5), and a modified version of this allele-specific primer, that is, (4) an allele-specific primer in which the second base from the 3′ end of the primer is the base corresponding to the target SNP base, and at least one base from the third base from the 3′ end to the 5′ end is introduced with a mismatch (for example, see Patent Document 6). Furthermore, there is also (5) an allele-specific primer in which the base corresponding to the target SNP base is located within four bases from the 3′ end, and a base adjacent to this base corresponding to the target SNP base is introduced with a mismatch the base type of which has been selected in accordance with the base type of the target SNP base (for example, see Patent Document 7).

Patent Document 1: Japanese Patent (Granted) Publication No. 2760553

Patent Document 2: Japanese Patent (Granted) Publication No. 2853864

Patent Document 3: United States Patent Application, Publication No. 2003/0022175 Specification

Patent Document 4: Japanese Patent (Granted) Publication No. 3859684

Patent Document 5: Japanese Unexamined Patent Application, First Publication No. 2005-287499

Patent Document 6: PCT International Publication No. 2001/042498 pamphlet

Patent Document 7: Japanese Unexamined Patent Application, First Publication No. 2004-121087

### DISCLOSURE OF INVENTION

### Problems to be Solved by the Invention

Regarding the allele-specific primers of Patent Documents 3 to 7, an attempt to solve the false positive problem has been made mainly by introducing a mismatch into a site adjacent to the base corresponding to the target SNP base so as to further unstabilize the vicinity of the SNP upon hybridization between the primer and a nucleic acid having the target nucleotide sequence to thereby improve the SNP discrimination ability of the allele-specific primer. However, even if the false positive can be kept low, in cases where the primer extension reaction efficiency largely varies depending on the allele, it is difficult to specify the allele on the basis of the degree of the primer extension reaction efficiency. For example, a complicated discrimination means such as a comparison of the primer extension reaction efficiency between respective samples with a control, has to be taken in many cases.

In response to such a problem, with the allele-specific primers of Patent Documents 4 and 7, an attempt not only to suppress the false positive but also to control the primer extension reaction efficiency has been made by introducing a mismatch of a specific base type. However, in Patent Document 4, the two-base combination to be introduced as a mismatch into the primer is determined only by focusing on the mismatch. In addition, in Patent Document 7, the base to be introduced as a mismatch is determined only by focusing on the base type of the target SNP base. For this reason, with these allele-specific primers, the primer extension reaction efficiency is not sufficiently controlled, depending on the type of the target nucleotide sequence, and it is difficult to specify the allele on the basis of the degree of the primer extension reaction efficiency.

It is an object of the present invention to provide a target base-specific primer for use in discrimination of the base type of a target base in a nucleic acid having a target nucleotide sequence including the target base in a nucleic acid sample, in the case where a plurality of base types can be enumerated for the target base, which is capable of discriminating the target base clearly with less possibility of false positive, and a target base discrimination method using the target base-specific primer.

### Means to Solve the Problems

In view of the above problems, the inventors of the present invention have conducted intensive studies. As a result, they have found that extremely high efficiency for a primer extension reaction can be achieved upon mismatch introduction into a base in a primer which corresponds to the target base (target base-corresponding base) by introducing a mismatch into a base adjacent to the 5′ side of the target base-corresponding base, and by setting the base type of a base to be introduced as a mismatch (mismatch base) to be a specific base type according to the combination between the base type of the target base and the base type of a base in the target nucleotide sequence which corresponds to the mismatch base (mismatch-corresponding base). This has led to the completion of the present invention.

That is, a first aspect of the present invention is a target base-specific primer for use in discrimination of the base type of a target base in a nucleic acid having a target nucleotide sequence including the target base in a nucleic acid sample, in the case where a plurality of base types can be enumerated for the target base, wherein the primer has a target base-corresponding base which is a base complementary to a base predicted as the target base, and a base type of the mismatch base is a base selected from the group consisting of the following (1a) to (1m) when the mismatch base refers to a base adjacent to the 5′ side of the target base-corresponding base, and a mismatch-corresponding base refers to a base adjacent to the 3′ side of the target base in the target nucleotide sequence:

(1a) if the base predicted as the target base is A and the mismatch-corresponding base is T, the mismatch base is either T or G;
(1b) if the base predicted as the target base is A and the mismatch-corresponding base is G, the mismatch base is T;
(1c) if the base predicted as the target base is T and the mismatch-corresponding base is T, the mismatch base is either T or G;
(1d) if the base predicted as the target base is T and the mismatch-corresponding base is G, the mismatch base is T;
(1e) if the base predicted as the target base is T and the mismatch-corresponding base is C, the mismatch base is A;
(1f) if the base predicted as the target base is G and the mismatch-corresponding base is A, the mismatch base is either A or C;
(1g) if the base predicted as the target base is G and the mismatch-corresponding base is T, the mismatch base is either T or G;
(1h) if the base predicted as the target base is G and the mismatch-corresponding base is G, the mismatch base is T;
(1i) if the base predicted as the target base is G and the mismatch-corresponding base is C, the mismatch base is any one of A, T, and C;
(1j) if the base predicted as the target base is C and the mismatch-corresponding base is A, the mismatch base is C;
(1k) if the base predicted as the target base is C and the mismatch-corresponding base is T, the mismatch base is either T or G;
(1l) if the base predicted as the target base is C and the mismatch-corresponding base is G, the mismatch base is A; and
(1m) if the base predicted as the target base is C and the mismatch-corresponding base is C, the mismatch base is C.

In addition, a second aspect of the present invention is a target base-specific primer for use in discrimination of the base type of a target base in a nucleic acid having a target nucleotide sequence including the target base in a nucleic acid sample, in the case where a plurality of base types can be enumerated for the target base, wherein the primer has a target base-corresponding base which is a base complementary to a base predicted as the target base, and a base type of the mismatch base is a base selected from the group consisting of the following (2a) to (2m) when the mismatch base refers to a base adjacent to the 5′ side of the target base-corresponding base, an anchor base refers to a base adjacent to the 3′ side of the mismatch-corresponding base, and a mismatch-corresponding base refers to a base adjacent to the 3′ side of the target base in the target nucleotide sequence:

(2a) if the anchor base is A and the mismatch-corresponding base is A, the mismatch base is C;
(2b) if the anchor base is A and the mismatch-corresponding base is T, the mismatch base is either G or C;
(2c) if the anchor base is A and the mismatch-corresponding base is C, the mismatch base is T;
(2d) if the anchor base is T and the mismatch-corresponding base is A, the mismatch base is C;
(2e) if the anchor base is T and the mismatch-corresponding base is A, the mismatch base is either G or C;
(2f) if the anchor base is T and the mismatch-corresponding base is C, the mismatch base is T;
(2g) if the anchor base is G and the mismatch-corresponding base is A, the mismatch base is either G or C;
(2h) if the anchor base is G and the mismatch-corresponding base is T, the mismatch base is either G or C;
(2i) if the anchor base is G and the mismatch-corresponding base is C, the mismatch base is either A or T;
(2j) if the anchor base is C and the mismatch-corresponding base is A, the mismatch base is any one of A, G, and C;
(2k) if the anchor base is C and the mismatch-corresponding base is T, the mismatch base is C;
(2l) if the anchor base is C and the mismatch-corresponding base is G, the mismatch base is any one of A, T, and G; and
(2m) if the anchor base is C and the mismatch-corresponding base is C, the mismatch base is either A or T.

In the first and second aspects of the present invention, if the mismatch base consists of one base, and if the base type of the mismatch base is different between the base selected from the group consisting of (1a) to (1m) and the base selected from the group consisting of (2a) to (2m) mentioned above, the mismatch base is preferably the base selected from the group consisting of (2a) to (2m).

Moreover, in the first and second aspects of the present invention, preferably, there are one or two of the mismatch base (s) and the mismatch-corresponding base(s).

Furthermore, in the first and second aspects of the present invention, the mismatch base is preferably located within four bases from the 3′ end.

In addition, a third aspect of the present invention is a method for discriminating the base type of a target base in a nucleic acid having a target nucleotide sequence including the target base in a nucleic acid sample, in the case where a plurality of base types can be enumerated for the target base, including the steps of: (I) hybridizing between the nucleic acid in the nucleic acid sample and the target base-specific primer of the first or second aspect of the present invention, and performing a nucleic acid extension reaction; and (II) discriminating the base type of the target base included in the nucleic acid in the nucleic acid sample on the basis of the nucleic acid extension efficiency of the step (I).

In the third aspect of the present invention, the step (II) is preferably a step (II-1) of determining that the base type of the target base included in the nucleic acid in the nucleic acid sample is the predicted base if the nucleic acid extension efficiency of the step (I) is high, and that the base type of the target base is different from the predicted base if the nucleic acid extension efficiency of the step (I) is low.

In addition, preferably, the method also includes, after the step (I) and before the step (II), a step (III) of hybridizing between the nucleic acid having a target nucleotide sequence in which the base type of the target base is different from the predicted base and the target base-specific primer, and performing a nucleic acid extension reaction, and the step (II) is a step (II-2) of determining that the base type of the target base included in the nucleic acid in the nucleic acid sample is the predicted base if the amount of the extension product resulting from the step (I) is greater than the amount of the extension product resulting from the step (III), and that the base type of the target base is different from the predicted base if the amount of the extension product resulting from the step (I) is not greater than the amount of the extension product resulting from the step (III). It is also preferable that the step (II-2) is a step (II-3) of determining that the base type of the target base included in the nucleic acid in the nucleic acid sample is the predicted base if the amount of the extension product resulting from the step (I) is three or more times greater than the amount of the extension product resulting from the step (III), and that the base type of the target base is different from the predicted base if the amount of the extension product resulting from the step (I) is less than three times the amount of the extension product resulting from the step (III).

In addition, in the third aspect of the present invention, the nucleic acid extension reaction is preferably a primer extension reaction.

### EFFECT OF THE INVENTION

The target base-specific primer of the first and second aspects of the present invention has a characteristic in which the discrimination ability for the target base is sufficiently high, and the nucleic acid extension reaction efficiency is sufficiently high upon a nucleic acid extension reaction after hybridization with a nucleic acid having a target nucleotide sequence including the target base. For this reason, the target base-specific primer of the present invention is suitably used particularly for discriminating the base type of the target base in a nucleic acid having a target nucleotide sequence including the target base in a nucleic acid sample, in the case where a plurality of base types can be enumerated for the target base.

Moreover, the target base can be discriminated clearly with less possibility of false positive by discriminating the base type of the target base with use of the target base discrimination method of the third aspect of the present invention.

### BRIEF DESCRIPTION OF THE DRAWINGS

FIG. 1 shows a form of hybridization between the target nucleic acid and the target base-specific primer serving as the first or second aspect of the present invention. FIG. 1 (a) shows a form of hybridization between the target base-specific primer and a target nucleic acid in which the base type of the target base is a specifically identifiable base for the target base-specific primer, and FIG. 1 (b) shows a form of hybridization between the target base-specific primer and a target nucleic acid in which the base type of the target base is not the specifically identifiable base for the target base-specific primer.

FIG. 2 shows the nucleotide sequence of the VKORC1 gene in the vicinity of VKORC1 (1173 C>T). The base C in the square is the base at the position 1173 serving as the detection subject.

FIG. 3 is a cluster diagram showing the detection results of VKORC1 (1173 C>T) in the <Detection of VKORC1 (1173 C>T) 1>. The y-axis shows the change in the A800 value of respective PCR-treated reaction solutions after the addition of anti-DIG dispersible fine particles and anti-FITC dispersible fine particles, and the x-axis shows the change in the A800 value of respective PCR-treated reaction solutions after the addition of anti-DNP dispersible fine particles and anti-FITC dispersible fine particles. The solid circle represents the result of PSCDA0182, the open circle represents the result of PSCDA0206, the solid triangle represents the result of PSCDA0009, and the mark x represents the result of the negative control.

FIG. 4 shows band patterns obtained in the <Detection of VKORC1 (1173 C>T) 2>. (a) shows the band pattern of the PCR product resulting from the VKORC1-C(T) primer and PSCDA0210, (b) shows the band pattern of the PCR product resulting from the VKORC1-C(T) primer and PSCDA0532, (c) shows the band pattern of the PCR product resulting from the VKORC1-C(A) primer and PSCDA0210, and (d) shows the band pattern of the PCR product resulting from the VKORC1-C(A) primer and PSCDA0532. The arrow A shows a band of the PCR product, and the arrows B and C show bands of markers.

### BRIEF DESCRIPTION OF THE REFERENCE SYMBOLS

1: Target base-specific primer, 2: Target nucleic acid

### BEST MODE FOR CARRYING OUT THE INVENTION

The target nucleotide sequence of the present invention is not specifically limited as long as the nucleotide sequence includes a target base serving as the subject of analysis and the nucleotide sequence has been elucidated to an analyzable degree by genetic recombination techniques or the like. Examples thereof may include nucleotide sequences existing in animal/plant chromosomes or bacterial/viral genes, and nucleotide sequences existing in biological RNAs such as mRNA. In the present invention, a nucleic acid having the target nucleotide sequence is referred to as a target nucleic acid.

In the present invention, the target base means a base which serves as the subject of analysis in a target nucleotide sequence, and a plurality of base types can be enumerated therefor. Here, the term “base type” means a type of nucleotide constituting a nucleotide sequence. Typically, a base portion of a nucleotide consists of any one of A (adenine), G (guanine), T (thymine), C (cytosine) when the target base is DNA, or any one of A, G, U (uracil), and C when the target base is RNA.

The target base in the present invention is not specifically limited as long as a plurality of base types can be enumerated therefor, although a gene polymorphism or a distinctive base in a disease marker gene is preferred. The term “distinctive base in a disease marker gene” means a base which enables discrimination of the nucleotide sequence of the marker gene from nucleotide sequences of other genes. Here, the term “gene polymorphism” is not specifically limited as long as the nucleotide sequence of a gene varies for the individual within a certain group of biological species. Examples of such gene polymorphism can include single nucleotide polymorphism (SNP) and microsatellite. In particular, the target base in the present invention is preferably SNP.

The nucleic acid sample in the present invention is not specifically limited as long as the sample is expected to contain a nucleic acid having the target nucleotide sequence (hereunder, may be referred to as the target nucleic acid).

Examples of the nucleic acid sample may include biological samples collected from an animal or the like, samples prepared from a cultured cell lysate or the like, and nucleic acid solutions extracted and purified from a biological sample or the like. In particular, human-derived biological samples to be used for clinical or other tests and nucleic acid solutions extracted and purified from such human-derived biological samples are preferred. In addition, the nucleic acid sample may be directly used after collection from an organism, or may be prepared before use. The preparation method is not specifically limited as long as a nucleic acid such as DNA or RNA contained in the biological sample is not impaired, and a usual preparation method for biological samples can be performed. Moreover, DNA extracted and purified from a biological sample and amplified by PCR or the like, and cDNA synthesized from RNA contained in a biological sample with a reverse transcriptase may also be used.

In the present invention, an attempt to solve the problem of false positive is made by introducing a mismatch serving as a non-complementary base to the target nucleic acid into a position adjacent to the target base, to thereby destabilize the vicinity of the target base in hybridization between the primer and the target nucleic acid, so as to improve the target base discrimination ability of the target base-specific primer. In particular, in the present invention, the target base discrimination ability is improved by optimizing the combination of the mismatch base and the base adjacent to the mismatch base in the primer.

Normally, the base type of the mismatch base may be any one of three types of non-complementary bases that are non-complementary to the base corresponding to the mismatch base in the target nucleic acid (hereunder, may be referred to as the mismatch-corresponding base). However, the portion of the mismatch base is unstable. From this unstable portion starts the separation of the overall primer from the target nucleic acid. Therefore, a base which should anchor the primer to the target nucleic acid, that is a base adjacent to the mismatch base, is important. Here, in the present invention, the base type of the mismatch base is set to be a specific base type which is optimum for the combination of the base type of the mismatch-corresponding base and the base type of the base adjacent to the mismatch-corresponding base in the target nucleic acid.

In the present invention, a base adjacent to the 5′ side of the target base-corresponding base in the target base-specific primer is set to be a mismatch base. That is, a base adjacent to the 3′ side of the target base in the target nucleotide sequence is a mismatch-corresponding base. That is to say, a specific base type which corresponds to the combination of the base type of the target base (base adjacent to the 5′ side of the mismatch-corresponding base) and the base type of the mismatch-corresponding base, or the combination of the base type of the base adjacent to the 3′ side of the mismatch-corresponding base (hereunder, may be referred to as an anchor base) and the base type of the mismatch-corresponding base, is introduced as the mismatch base.

Specifically, the target base-specific primer serving as the first aspect of the present invention is a primer for use in discrimination of the base type of a target base in a nucleic acid having a target nucleotide sequence including the target base in a nucleic acid sample, in the case where a plurality of base types can be enumerated for the target base, wherein the primer has a target base-corresponding base which is a base complementary to a base predicted as the target base, and the base type of a mismatch base is a base selected from the group consisting of the following (1a) to (1m) when the mismatch base refers to a base adjacent to the 5′ side of the target base-corresponding base, and a mismatch-corresponding base refers to a base adjacent to the 3′ side of the target base in the target nucleotide sequence:

(1a) if the base predicted as the target base is A and the mismatch-corresponding base is T, the mismatch base is either T or G;
(1b) if the base predicted as the target base is A and the mismatch-corresponding base is G, the mismatch base is T;
(1c) if the base predicted as the target base is T and the mismatch-corresponding base is T, the mismatch base is either T or G;
(1d) if the base predicted as the target base is T and the mismatch-corresponding base is G, the mismatch base is T;
(1e) if the base predicted as the target base is T and the mismatch-corresponding base is C, the mismatch base is A;
(1f) if the base predicted as the target base is G and the mismatch-corresponding base is A, the mismatch base is either A or C;
(1g) if the base predicted as the target base is G and the mismatch-corresponding base is T, the mismatch base is either T or G;
(1h) if the base predicted as the target base is G and the mismatch-corresponding base is G, the mismatch base is T;
(1i) if the base predicted as the target base is G and the mismatch-corresponding base is C, the mismatch base is either T or C,
(1j) if the base predicted as the target base is C and the mismatch-corresponding base is A, the mismatch base is C;
(1k) if the base predicted as the target base is C and the mismatch-corresponding base is T, the mismatch base is either T or G;
(1l) if the base predicted as the target base is C and the mismatch-corresponding base is G, the mismatch base is A; and
(1m) if the base predicted as the target base is C and the mismatch-corresponding base is C, the mismatch base is C.

In addition, the target base-specific primer serving as the second aspect of the present invention is a primer for use in discrimination of the base type of a target base in a nucleic acid having a target nucleotide sequence including the target base in a nucleic acid sample, in the case where a plurality of base types can be enumerated for the target base, wherein the primer has a target base-corresponding base which is a base complementary to a base predicted as the target base, and the base type of a mismatch base is a base selected from the group consisting of the following (2a) to (2m) when the mismatch base refers to a base adjacent to the 5′ side of the target base-corresponding base, an anchor base refers to a base adjacent to the 3′ side of the mismatch-corresponding base, and a mismatch-corresponding base refers to a base adjacent to the 3′ side of the target base in the target nucleotide sequence:

(2a) if the anchor base is A and the mismatch-corresponding base is A, the mismatch base is C;
(2b) if the anchor base is A and the mismatch-corresponding base is T, the mismatch base is either G or C;
(2c) if the anchor base is A and the mismatch-corresponding base is C, the mismatch base is T;
(2d) if the anchor base is T and the mismatch-corresponding base is A, the mismatch base is C;
(2e) if the anchor base is T and the mismatch-corresponding base is T, the mismatch base is either G or C;
(2f) if the anchor base is T and the mismatch-corresponding base is C, the mismatch base is T;
(2g) if the anchor base is G and the mismatch-corresponding base is A, the mismatch base is either G or C;
(2h) if the anchor base is G and the mismatch-corresponding base is T, the mismatch base is either G or C;
(2i) if the anchor base is G and the mismatch-corresponding base is C, the mismatch base is either A or T;
(2j) if the anchor base is C and the mismatch-corresponding base is A, the mismatch base is any one of A, G, and C;
(2k) if the anchor base is C and the mismatch-corresponding base is A, the mismatch base is C;
(2l) if the anchor base is C and the mismatch-corresponding base is G, the mismatch base is any one of A, T, and G; and
(2m) if the anchor base is C and the mismatch-corresponding base is C, the mismatch base is either A or T.

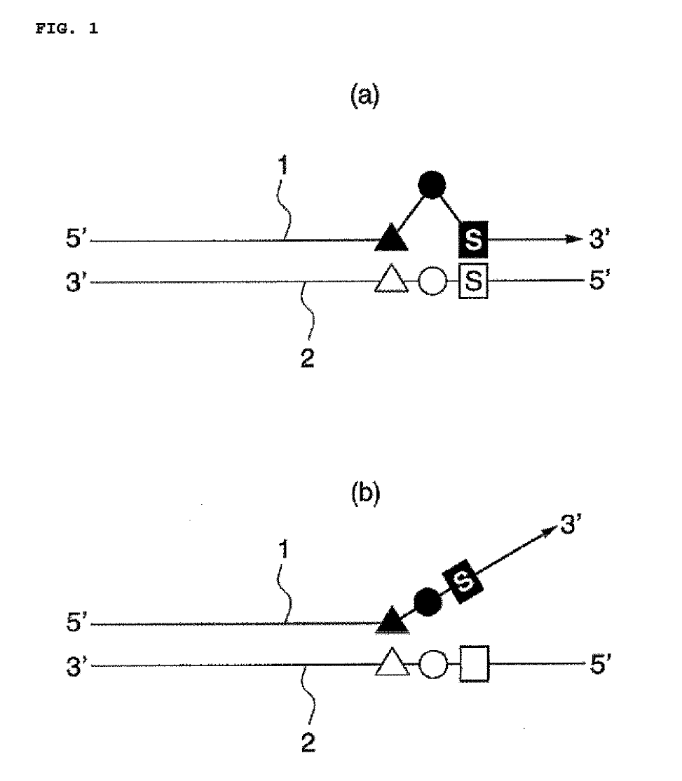

FIG. 1 shows a form of hybridization between the target nucleic acid and the target base-specific primer serving as the first or second aspect of the present invention. FIG. 1 (a) shows a form of hybridization between the target base-specific primer and a target nucleic acid in which the base type of the target base is a specifically identifiable base for the target base-specific primer, and FIG. 1 (b) shows a form of hybridization between the target base-specific primer and a target nucleic acid in which the base type of the target base is not the specifically identifiable base for the target base-specific primer. In the target nucleic acid 2, the letter S in the square means a target base whose base type is as predicted, the open square without the letter S means a target base whose base type is not the predicted one, the open circle means a mismatch-corresponding base, and the open triangle means an anchor base. In addition, in the target base-specific primer 1, the letter S in the solid square means a target base-corresponding base, the solid circle means a mismatch base, and the solid triangle means a base corresponding to the anchor base.

That is, in the target base-specific primer of the first aspect of the present invention, for example, when the target base S (in open square) is A and the mismatch-corresponding base (open circle) is T in the target nucleotide sequence, the mismatch base (solid circle) is set as T or G. In addition, when the target base S (in open square) is C and the mismatch-corresponding base (open circle) is C in the target nucleotide sequence, the mismatch base (solid circle) is set as C. On the other hand, in the target base-specific primer of the second aspect of the present invention, for example, when the anchor base (open triangle) is A and the mismatch-corresponding base (open circle) is A in the target nucleotide sequence, the mismatch base (solid circle) is set as C. In addition, when the anchor base (open triangle) is C and the mismatch-corresponding base (open circle) is C in the target nucleotide sequence, the mismatch base (solid circle) is set as A or T.

In the first and second aspects, in the case where a plurality of base types can be enumerated for the mismatch base, any base type may be selected. For example, the base type can be appropriately selected by considering the Tm value and the base length of the target base-specific primer, the type of another primer to be used together, the reaction condition for the nucleic acid extension reaction, and the like.

Moreover, either one of the target base-specific primer of the first aspect and the target base-specific primer of the second aspect may be used. The type of the target base-specific primer among those two of the first aspect and the second aspect can be appropriately selected by considering the type of the target nucleotide sequence, the reaction condition for the nucleic acid extension reaction, and the like.

By employing a target base-specific primer combined with the first aspect and the second aspect, a preferable effect can be given in which the base type of the target base can be more accurately discriminated. For example, when the target base is A, the mismatch-corresponding base is T, and the anchor base is A in the target nucleotide sequence, then T or G can be employed as the mismatch base in the target base-specific primer of the first aspect (1a), and G or C can be employed as the mismatch base in the target base-specific primer of the second aspect (2b). Accordingly, by employing G as the mismatch base, the target base-specific primer which enables the most accurate discrimination of the base type of the target base can be obtained.

If the mismatch base consists of one base, the selected base type of the mismatch base may be different between the first aspect and the second aspect. In this case, the priority is preferably given to the second aspect. That is, if the mismatch base consists of one base, and if the base type of the mismatch base is different between the base selected from the group consisting of (1a) to (1m) and the base selected from the group consisting of (2a) to (2m) as mentioned above, the base type of the mismatch base is preferably set to be the base selected from the group consisting of (2a) to (2m).

For example, when the base predicted as the target base is C, the mismatch-corresponding base is C, and the anchor base is T, then the rule (1m) in the first aspect instructs that the mismatch base is C, whereas the rule (2f) in the second aspect instructs that the mismatch base is T. Therefore, the base type of the mismatch base is incompatible. In this case, the mismatch base of the target base-specific primer is preferably set to be T rather than C.

In the target base-specific primer of the first aspect and/or the second aspect (hereunder, may be referred to as the target base-specific primer of the present invention), the target base-corresponding base, the mismatch base, and the base corresponding to the anchor base have only to be located in this order from the 3′ end side of the primer. That is, the position of the target base-corresponding base in the target base-specific primer is not necessarily limited, although it is preferable that the mismatch base be located within four bases from the 3′ end, that is, the target base-corresponding base is located within three bases from the 3′ end of the target base-specific primer. The reason is that the target base discrimination ability of the primer can be sufficiently improved by locating the target base-corresponding base and the mismatch base in the vicinity of the 3′ end of the target base-specific primer. More preferably, the target base-corresponding base and the mismatch base are respectively located at the second and the third base from the 3′ end of the target base-specific primer.

In addition, in the target base-specific primer of the present invention, the number of mismatch bases is not specifically limited as long as the mismatch base(s) is (are) adjacent to the 5′ side of the target base-corresponding base. For example, consecutive bases one end of which is adjacent to the 5′ side of the target base-corresponding base may be used as the mismatch bases. The number of mismatch bases is preferably one or two, and more preferably one.

In the target base-specific primer of the present invention, the bases other than the target base-corresponding base, the mismatch base, and the base corresponding to the anchor base, are preferably bases complementary to the target nucleotide sequence, although they are not specifically limited as long as the base type of the target base can be discriminated by using the target base-specific primer of the present invention, and they do not have to be completely complementary to the target nucleotide sequence.

The target base-specific primer of the present invention can be designed according to the target nucleotide sequence by a usual method. For example, it can be easily designed by using the nucleotide sequence information available from publicly known genome sequence data or SNP data with a general primer design tool. The publicly known genome sequence data is usually available on international nucleotide sequence databases, NCBI (National Center for Biotechnology Information), DDBJ (DNA Data Bank of Japan), and the like. In addition, the publicly known SNP data is available on databases such as a Japanese SNP database, JSNP (http://snp.ims.u-tokyo.ac.jp/index_ja.html) constructed by the Institute of Medical Science, the University of Tokyo. Examples of the primer design tool include Primer3 (Rozen, S., H. J. Skaletsky (1996), http://www-genome.wi.mit.edu/genome_software/other/primer3.html) and Visual OMP (DNA Software) which are available on the web.

The thus designed primer can be synthesized by any method well known in the art. For example, it may be synthesized by a custom oligo synthesis service or may be synthesized by the user themselves using a commercially available synthesizer.

Moreover, the target base-specific primer of the present invention may have an additional sequence besides the region which hybridizes with the target nucleic acid, to an extent that the discrimination of the base type of the target base is not inhibited. Examples of such an additional sequence include restriction enzyme recognition sequences and sequences for labeling a nucleic acid.

Furthermore, the target base-specific primer of the present invention may be labeled in order to facilitate the detection, analysis, and the like of the nucleic acid extension product resulting from the step (I) that will be described later. The labeling substance is not specifically limited as long as it can be used for labeling nucleic acids. Examples thereof include radioisotopes, fluorescent substances, chemiluminescent substances, and biotin.

The target base discrimination method of the third aspect of the present invention (hereunder, may be referred to as the target base discrimination method of the present invention) is a method for discriminating the base type of a target base with use of the target base-specific primer of the present invention. Specifically, the target base discrimination method of the present invention is a method for discriminating the base type of a target base in a nucleic acid having a target nucleotide sequence including the target base in a nucleic acid sample, in the case where a plurality of base types can be enumerated for the target base, including the steps of: (I) hybridizing the nucleic acid in the nucleic acid sample and the target base-specific primer of the present invention, and performing a nucleic acid extension reaction; and (II) discriminating the base type of the target base included in the nucleic acid in the nucleic acid sample on the basis of the nucleic acid extension efficiency of the step (I).

For example, in FIG. 1 (a), the target base-specific primer 1 and the target nucleic acid 2 are complementary to each other except for the mismatch base in the target base-specific primer 1. That is, the 3′ end side of the target base-specific primer 1 hybridizes with the target nucleic acid 2, and thus the nucleic acid extension reaction of the target base-specific primer 1 with the target nucleic acid 2 as a template efficiently progresses. On the other hand, in FIG. 1 (b), the target base-specific primer 1 and the target nucleic acid 2 are non-complementary at two bases, namely, the mismatch base and the target base-corresponding base, in the target base-specific primer 1. For this reason, the 3′ end side of the target base-specific primer 1 hardly hybridizes with the target nucleic acid 2, and the nucleic acid extension reaction efficiency of the target base-specific primer 1 with the target nucleic acid 2 as a template is lowered. In this manner, in the step (II), it can be determined that the base type of the target base included in the nucleic acid in the nucleic acid sample is the predicted base (a base complementary to the target base-corresponding base of the target base-specific primer used in the nucleic acid extension reaction) if the nucleic acid extension efficiency of the step (I) is high, and that the base type of the target base is different from the predicted base if the nucleic acid extension efficiency of the step (I) is low.

Here, the degree of the nucleic acid extension reaction efficiency can be appropriately determined by considering the type of the nucleic acid extension reaction, the reaction condition, and the like, by those skilled in the art. For example, the determination can be done on the basis of the nucleic acid extension reaction efficiency resulting from a nucleic acid extension reaction performed in the same manner as that of the step (I) with use of a target nucleic acid, in which the base type of the target base is known in advance, as a template.

Specifically, the method may also comprise, after the step (I) and before the step (II), the step (III) of hybridizing the nucleic acid having a target nucleotide sequence in which the base type of the target base is different from the predicted base and the target base-specific primer, and performing a nucleic acid extension reaction, and then it can be determined in the step (II) that the base type of the target base included in the nucleic acid in the nucleic acid sample is the predicted base if the amount of the extension product resulting from the step (I) is greater than the amount of the extension product resulting from the step (III), and that the base type of the target base is different from the predicted base if the amount of the extension product resulting from the step (I) is not greater than the amount of the extension product resulting from the step (III).

In addition, as will be described later, when the extension reaction is performed by PCR method such as real time PCR or another PCR method, a technique to compare the extension reaction efficiency on the basis of the number of cycles is generally employed. In this case, for example, it can be determined that extension product amounts are different if the ratio of these product amounts corresponds to at least about two cycles. Here, assuming that the amplification rate per cycle is 1.75, the product amount ratio for two cycles is about 3 (1.75×1.75≅3.06). For this reason, it is also preferable to determine in the step (II) that the base type of the target base included in the nucleic acid in the nucleic acid sample is the predicted base if the amount of the extension product resulting from the step (I) is three or more times greater than the amount of the extension product resulting from the step (III), and that the base type of the target base is different from the predicted base if the amount of the extension product resulting from the step (I) is less than three times the amount of the extension product resulting from the step (III).

The nucleic acid extension reaction in the step (I) is not specifically limited as long as the reaction is performed by using the hybridization between the nucleic acid in the nucleic acid sample and the target base-specific primer of the present invention. Such a reaction can be appropriately selected from usual nucleic acid extension reactions performed in the field of genetic analysis and the like. For example, there may also be employed a primer extension reaction or a reaction for linking with another primer using a ligase.

In the target base discrimination method of the present invention, the primer extension reaction is preferably used as the nucleic acid extension reaction. The primer extension reaction is not specifically limited as long as it is a reaction to extend the target base-specific primer of the present invention with the nucleic acid in the nucleic acid sample as a template. Such a reaction can be appropriately selected from usual nucleic acid extension reactions performed in the field of genetic analysis and the like. In the target base discrimination method of the present invention, nucleic acid amplification reactions such as a PCR (Polymerase Chain Reaction) method, a LAMP (Loop-Mediated Isothermal Amplification) method, a SMAP (SMart Amplification Process) method, a RCR (Recombinatorial Chain Reaction) method, a TMA (Transcripition mediated amplification) method, a PALSAR (Probe alternation link self-assembly reaction) method, a NASBA (nucleic acid sequence-based amplification) method, and an ICAN (Isothermal and chimeric primer-initiated amplification of nucleic acids) method are more preferred as the primer extension reaction since these methods are capable of discriminating the base type of the target base even if the amount of the target nucleic acid in the nucleic acid sample is very small.

If a nucleic acid amplification reaction such as a PCR method is employed as the nucleic acid extension reaction in the step (I), the nucleic acid extension efficiency of the step (I) can be examined on the basis of the amount of the amplification product yielded from the nucleic acid amplification reaction. The reason is that the amount of the amplification product is large if the nucleic acid extension efficiency is high, while the amount of the amplification product is small if the nucleic acid extension efficiency is low.

Moreover, the nucleic acid extension efficiency can also be examined on the basis of the molecular weight of the amplification product yielded from the nucleic acid amplification reaction. The reason is that since the target base discrimination method of the present invention uses the target base-specific primer of the present invention having a very high target base discrimination ability, the nucleic acid extension reaction efficiency is very low if the base type of the target base included in the nucleic acid in the nucleic acid sample is non-complementary to the target base-corresponding base of the target base-specific primer, and this causes a remarkably low possibility of false positive, so that it can be determined that the nucleic acid extension efficiency is high if the molecular weight of the amplification product meets the predicted molecular weight.

Enzymes such as a polymerase, reagents such as nucleotides and a buffer for use in the nucleic acid extension reaction are not specifically limited, and those for general use can be used at usual concentrations. In addition, the nucleic acid extension reaction can be performed by a usual method, and the reaction condition and the like can be appropriately determined by considering the type of the target nucleotide sequence, the type and amount of the nucleic acid sample, the type of an enzyme to be used, and the like.

For example, the following procedure can be followed to perform the primer extension reaction. First, the target base-specific primer of the present invention, a polymerase, nucleotides, a buffer, and a desired salt or the like as needed, are added to the nucleic acid sample to prepare a reaction solution. This reaction solution is placed under a high temperature condition so as to denature the double stranded nucleic acid in the nucleic acid sample. Then, the temperature is lowered so as to form a double stranded nucleic acid between the nucleic acid and the target base-specific primer of the present invention. Thereafter, the temperature condition is set so that the DNA polymerase can perform the extension reaction. By so doing, the primer extension reaction is efficiently performed from the target base-specific primer when the double stranded nucleic acid in the nucleic acid sample has a target base whose base type is complementary to the target base-corresponding base in the target base-specific primer. Conversely, the efficiency of the primer extension reaction from the target base-specific primer is remarkably impaired when the double stranded nucleic acid in the nucleic acid sample has a target base whose base type is non-complementary to the target base-corresponding base in the target base-specific primer. Accordingly, the base type of the target base can be discriminated clearly with much less possibility of false positive by examining the primer extension reaction efficiency during or after this primer extension reaction.

The method for measuring the primer extension reaction efficiency is not specifically limited, and the efficiency can be measured by a usual method. For example, the primer extension reaction efficiency can be measured by measuring the amount of pyrophosphate released as a result of the primer extension reaction. The method for measuring the pyrophosphate amount is not specifically limited, and a usual measurement method can be taken such as methods proposed in Nucleic Acids Research, Vol. 9, No. 19 e93 (2001), PCT International Publication No. WO 2003/078655 pamphlet, and Japanese Unexamined Patent Application, First Publication No. 2004-141158. Moreover, the primer extension reaction efficiency may be examined by carrying out the primer extension reaction in the state where the 5′ end of the target base-specific primer is immobilized on a crystal oscillator, and monitoring the primer extension reaction through analysis of the change in its oscillation frequency. Alternatively, the primer extension reaction efficiency may also be examined by carrying out the aforementioned primer extension reaction in the state where the 5′ end of the target base-specific primer is immobilized on a gold surface, and monitoring the primer extension reaction through analysis of the surface plasmon resonance (SPR) phenomenon.

The specific procedure for carrying out the primer extension reaction is not limited to the above-mentioned procedures. For example, the primer extension reaction may be performed after all the necessary reagents are previously mixed in the reaction mixture, similar to the procedure described above. Moreover the primer extension reaction may be performed by a so-called hot start method in which the target base-specific primer of the present invention, a buffer, and a desired salt or the like as needed are firstly added to the nucleic acid sample to previously prepare a solution, the solution is subjected to a denaturation step at a high temperature and formation of a double stranded nucleic acid with the target base-specific primer is performed, after which the solution is serially added with a polymerase and nucleotides to execute the reaction. Moreover, in cases where the nucleic acid in the nucleic acid sample is a single stranded nucleic acid, a step of placing under a high temperature condition, meaning to allow the nucleic acid to be denatured is not required, unlike in the case of the double stranded nucleic acid. However, the single stranded nucleic acid and the target base-specific primer must be conditioned to provide a state in which both of them are sufficiently dissociated, meaning to control the reaction to form the double strand between the single stranded nucleic acid and the target base-specific primer. Therefore, it is more preferred that a step of placing them under a high temperature condition is conducted prior to the step of forming the double strand between the target base-specific primer and the nucleic acid in the nucleic acid sample.

In addition, the polymerase for use in the primer extension reaction is not specifically limited, and may be a usual polymerase in the field of genetic analysis and the like.

For example, either DNA polymerase or RNA polymerase may be used. Moreover, the polymerase may or may not be heat-resistant. Furthermore, the polymerase may or may not have a 3′->5′ exonuclease activity. In addition, the polymerase may have a strand exchange activity.

In the target base discrimination method of the present invention, it is preferred that the polymerase particularly has a weak or no 3′->5′ exonuclease activity. In the present invention, when the primer extension reaction is performed using a polymerase having no 31->5′ exonuclease activity, the reaction progresses highly efficiently if the nucleic acid in the nucleic acid sample has a target base whose base type is complementary to the target base-corresponding base in the target base-specific primer, whereas the primer extension reaction hardly progresses if the nucleic acid in the nucleic acid sample has a target base whose base type is non-complementary to the target base-corresponding base in the target base-specific primer. Accordingly, the reason is that the possibility of false positive can be remarkably lowered by using a polymerase having a weak or no 3′->5′ exonuclease activity. However, the target base discrimination method of the present invention does not always require use of a polymerase having no 3′->5′ exonuclease activity. For example, the base type of the target base can be discriminated with high accuracy even with a KOD DNA polymerase derived from the hyperthermophilic archaeon Thermococcus kodakaraensis KOD1 having a 3′->5′ exonuclease activity.

In the target base discrimination method of the present invention, it is particularly preferred to perform PCR with use of the target base-specific primer of the present invention and the target nucleic acid as a template, as the primer extension reaction. Specifically, the PCR can be performed by the following manner: the nucleic acid sample is added with the target base-specific primer of the present invention, a second primer having a relation of forward primer/reverse primer with respect to this target base-specific primer, a DNA polymerase for PCR, nucleotides, Mg ion, a buffer, and a desired salt or the like as needed, to prepare a reaction solution; then, the reaction solution is subjected to 1 to 40 cycles consisting of a denaturation step, an annealing step, and an extension step by a usual method. A nucleic acid in a region between the target base-specific primer of the present invention and the second primer in the target nucleic acid is efficiently amplified when the double stranded nucleic acid in the nucleic acid sample has a target base whose base type is complementary to the target base-corresponding base in the target base-specific primer. Conversely, the nucleic acid in the region is little amplified when the double stranded nucleic acid in the nucleic acid sample has a target base whose base type is non-complementary to the target base-corresponding base in the target base-specific primer. Accordingly, the base type of the target base can be discriminated clearly with much less possibility of false positive by examining the amount of the nucleic acid in the region serving as the amplification product, during or after PCR.

The method for measuring the amount of the amplification product is not specifically limited, and the amount can be measured by a usual method for the measurement of PCR products. For example, as described above, the amount of pyrophosphate released as a result of PCR may be measured. The measurement can also be done using the electrophoretic method. In addition, the measurement can be done by a method for measuring the fluorescence intensity with a fluorescent intercalator.

Moreover, by performing PCR using the target base-specific primer previously labeled with an appropriate fluorescent substance, the amount, the molecular weight, and so on of the resultant nucleic acid amplification product can be analyzed through single molecule fluorescence detection. In the single molecule fluorescence detection, the resultant nucleic acid amplification product is analyzed on the basis of the change in the molecular diffusion rate. More specifically, a nucleic acid in a region between the target base-specific primer and the above-mentioned second primer is efficiently amplified when the double stranded nucleic acid in the nucleic acid sample has a target base whose base type is complementary to the target base-corresponding base in the target base-specific primer. Therefore, the molecular weight of the fluorescence-labeled target base-specific primer is greatly increased. Since the diffusion rate of a substance in a solution depends on the molecular weight of the substance, the diffusion rate of the fluorescence-labeled target base-specific primer is remarkably lowered. On the other hand, the nucleic acid in the region between the target base-specific primer and the above-mentioned second primer is little amplified when the double stranded nucleic acid in the nucleic acid sample has a target base whose base type is non-complementary to the target base-corresponding base in the target base-specific primer. Therefore, the molecular weight of the fluorescence-labeled target base-specific primer remains unchanged, and the diffusion rate thereof is almost unchanged. That is, the presence or absence of the extension of the target base-specific primer can be examined by measuring the diffusion rate of the PCR-resultant nucleic acid amplification product through the single molecule fluorescence detection. On the basis of this result, the base type of the target base can be discriminated. The analysis method through the single molecule fluorescence detection is not specifically limited, and can be performed using a usual analysis method such as the method described in Nature Genetics, Vol. 38, pp. 324 to 330 (2006). In the analysis method through the single molecule fluorescence detection, sufficient difference in the molecular weight per molecule has only to be made, and even a very small amount of the nucleic acid amplification product is analyzable. Therefore, for example, the base type of the target base can be discriminated even if the extension reaction is performed using the labeled target base-specific primer alone without the above-mentioned second primer. Moreover, multiplex PCR detection may also be performed based on a usual method by adding a plurality of types of target base-specific primers labeled with different fluorescent substances in one solution.

In addition, the nucleic acid amplification product resulting from the nucleic acid amplification reaction such as PCR can also be detected on the basis of the aggregation of microbeads. In the detection method on the basis of the aggregation of microbeads, PCR is performed by a usual method using the target base-specific primer of the present invention modified with a first ligand and the above-mentioned second primer modified with a second ligand of a different type from the first ligand, with the nucleic acid in the nucleic acid sample as a template, followed by detection of the resultant amplification product using first microbeads bound with a receptor which is specifically bindable to the first ligand and second microbeads bound with a receptor which is specifically bindable to the second ligand. The resultant amplification product will have both ends respectively modified with the first ligand and the second ligand if the double stranded nucleic acid in the nucleic acid sample has a target base whose base type is complementary to the target base-corresponding base in the target base-specific primer. Therefore, when the amplification product is mixed with the first microbeads and the second microbeads, these microbeads form a cross-linked structure via the amplification product. A large number of occurrences of this reaction makes these microbeads aggregated. On the other hand, both ends of the resultant amplification product will not be modified if the double stranded nucleic acid in the nucleic acid sample has a target base whose base type is non-complementary to the target base-corresponding base in the target base-specific primer. Therefore, even if the amplification product is mixed with the first microbeads and the second microbeads, these microbeads are not aggregated. The reason is that a primer by itself can cause the ligand-receptor reaction but does not cause the cross-linkage, so the aggregation will not occur without the presence of the amplification product having both ends modified with different ligands. Accordingly, the base type of the target base can be discriminated by detecting the presence or absence of the aggregation of microbeads. This detection method on the basis of the aggregation of microbeads is not specifically limited, and can be performed by using a usual analysis method such as the method described in Japanese Unexamined Patent Application, First Publication No. 2005-95134.

### EXAMPLES

Next is a more detailed description of the present invention with reference to Examples. However, the present invention is not to be considered as being limited by the following Examples.

### Preparation of Primers for Producing Template DNA

The template DNA was made by mutagenesis based on the pUC19 DNA (manufactured by TaKaRa Bio) with the GeneTailor Site-Directed Mutagenesis System (manufactured by Invitrogen). Primers used for the mutagenesis consisted of a mutated forward primer and a normal reverse primer. The reverse primer used herein was the Mi-R that was in common to every forward primer. Moreover, the forward primers were divided into two major groups consisting of the Mi-F-uXX (XX stands for a combination of two types of bases) series for examining the influence of the mismatch from the 3′ side, and the Mi-F-dXX (XX stands for a combination of two types of bases) series for examining the influence from the 5′ side. Each pUC19 DNA was differentially mutated by respective combination of the forward primer and the reverse primer. The details of these primers are shown in Table 1. In the table, the underlined bases in each sequence show the mutation site. For example, the mutagenesis PCR with Mi-F-uAA and Mi-R provided a mutant in which the target base was A and the mismatch base was A.

TABLE 1

| | |
|---|---|
| primer | Sequence |

| | |
|---|---|
| Mi-F-uAA | 5′-TTAAGGGATTTTGGTCATGAGAATATCAAAAAGGA-3′ |
| Mi-F-uAT | 5′-TTAAGGGATTTTGGTCATGAGATTATCAAAAAGGA-3′ |
| Mi-F-uAG | 5′-TTAAGGGATTTTGGTCATGAGAGTATCAAAAAGGA-3′ |
| Mi-F-uAC | 5′-TTAAGGGATTTTGGTCATGAGACTATCAAAAAGGA-3′ |
| Mi-F-uTA | 5′-TTAAGGGATTTTGGTCATGAGTATATCAAAAAGGA-3′ |
| Mi-F-uTT | 5′-TTAAGGGATTTTGGTCATGAGTTTATCAAAAAGGA-3′ |
| Mi-F-uTG | 5′-TTAAGGGATTTTGGTCATGAGTGTATCAAAAAGGA-3′ |
| Mi-F-uTC | 5′-TTAAGGGATTTTGGTCATGAGTCTATCAAAAAGGA-3′ |
| Mi-F-uGA | 5′-TTAAGGGATTTTGGTCATGAGGATATCAAAAAGGA-3′ |
| Mi-F-uGT | 5′-TTAAGGGATTTTGGTCATGAGGTTATCAAAAAGGA-3′ |
| Mi-F-uGG | 5′-TTAAGGGATTTTGGTCATGAGGGTATCAAAAAGGA-3′ |
| Mi-F-uGC | 5′-TTAAGGGATTTTGGTCATGAGGCTATCAAAAAGGA-3′ |
| Mi-F-uCA | 5′-TTAAGGGATTTTGGTCATGAGCATATCAAAAAGGA-3′ |
| Mi-F-uCT | 5′-TTAAGGGATTTTGGTCATGAGCTTATCAAAAAGGA-3′ |
| Mi-F-uCG | 5′-TTAAGGGATTTTGGTCATGAGCGTATCAAAAAGGA-3′ |
| Mi-F-uCC | 5′-TTAAGGGATTTTGGTCATGAGCCTATCAAAAAGGA-3′ |
| Mi-F-dAA | 5′-TTAAGGGATTTTGGTCATGAAATTATCAAAAAGGA-3′ |
| Mi-F-dAT | 5′-TTAAGGGATTTTGGTCATGAATTTATCAAAAAGGA-3′ |
| Mi-F-dAG | 5′-TTAAGGGATTTTGGTCATGAAGTTATCAAAAAGGA-3′ |
| Mi-F-dAC | 5′-TTAAGGGATTTTGGTCATGAACTTATCAAAAAGGA-3′ |
| Mi-F-dTA | 5′-TTAAGGGATTTTGGTCATGATATTATCAAAAAGGA-3′ |
| Mi-F-dTT | 5′-TTAAGGGATTTTGGTCATGATTTTATCAAAAAGGA-3′ |
| Mi-F-dTG | 5′-TTAAGGGATTTTGGTCATGATGTTATCAAAAAGGA-3′ |
| Mi-F-dTC | 5′-TTAAGGGATTTTGGTCATGATCTTATCAAAAAGGA-3′ |
| Mi-F-dGA | 5′-TTAAGGGATTTTGGTCATGAGATTATCAAAAAGGA-3′ |
| Mi-F-dGT | 5′-TTAAGGGATTTTGGTCATGAGTTTATCAAAAAGGA-3′ |
| Mi-F-dGG | 5′-TTAAGGGATTTTGGTCATGAGGTTATCAAAAAGGA 3′ |
| Mi-F-dGC | 5′-TTAAGGGATTTTGGTCATGAGCTTATCAAAAAGGA-3′ |
| Mi-F-dCA | 5′-TTAAGGGATTTTGGTCATGACATTATCAAAAAGGA-3′ |
| Mi-F-dCT | 5′-TTAAGGGATTTTGGTCATGACTTTATCAAAAAGGA-3′ |
| Mi-F-dCG | 5′-TTAAGGGATTTTGGTCATGACGTTATCAAAAAGGA-3′ |
| Mi-F-dCC | 5′-TTAAGGGATTTTGGTCATGACCTTATCAAAAAGGA-3′ |
| Mi-R | 5′-ACGAAAACTCACGTTAAGGGATTTTGGTCATGA-3′ |

### Methylation

In accordance with the protocol of the GeneTailor Site-Directed Mutagenesis System (manufactured by Invitrogen), separately purchased pUC19 DNA (manufactured by TaKaRa Bio) was methylated. Specifically, 100 ng of pUC19 DNA, 1.6 μL of 10×SAM, 1.0 μL of DNA methylase (4 U/μL), and an appropriate amount of sterile water were added to 1.6 μL of 10× methylation buffer to prepare 16 μL of a reaction solution, followed by incubation at 37° C. for 1 hour.

### Mutagenesis PCR

Mutagenesis PCR was performed on the methylated pUC19 DNA mentioned above with the respective mutagenesis primers mentioned above. Basically, the mutagenesis PCR was performed using the GeneTailor Site-Directed Mutagenesis System (manufactured by Invitrogen). Specifically, 1.5 μL of 10 mM dNTPs, 1 μL of 50 mM magnesium sulfate, 1.5 μL of 10 μM forward primer, 1.5 μL of 10 μM reverse primer, 12.5 ng of methylated DNA, 0.2 μL of Platinum Taq High Fidelity (5 U/μL), and an appropriate amount of sterile water were added to 5 μL of 10× High Fidelity PCR buffer to prepare 50 μL of a reaction solution. Then, the reaction solution was subjected to the mutagenesis PCR by treatment at 94° C. for 2 minutes, subsequent 24 thermal cycles at 94° C. for 30 seconds, 55° C. for 30 seconds, and 68° C. for 3 minutes, and an additional treatment at 68° C. for 10 minutes. The PCR apparatus was the Gradient Thermal Cycler PTC-200 manufactured by the former MJ Research, Inc. (currently, Bio-Rad Laboratories).

### Transformation

The mutated product resulting from the above-mentioned mutagenesis PCR was transformed. Basically, the transformation was performed in accordance with the protocol of the GeneTailor Site-Directed Mutagenesis System (manufactured by Invitrogen). The competent cells used herein were the One-Shot (registered trademark) Max Efficiency DH5α (registered trademark)-T1R competent cells provided in the kit of this System. The LB agar used herein was the FastMedia (registered trademark) AMP X-GAL (manufactured by CAYLA), an ampicillin-containing LB medium. Specifically, the DH5α-T1R competent cells lysed on ice were directly added with 2 μL of the reaction solution after the mutagenesis PCR as mentioned above, followed by tapping and incubation on ice for 10 minutes. Thereafter, the mixture was incubated in a water bath at 42° C. for 30 seconds, and immediately transferred onto ice and left for 1 minute. Then, 200 μL of SOC medium was added thereto. The SOC medium had been placed at room temperature in advance. The mixture was further incubated at 37° C. for 1 hour under shaking at 125 rpm. This competent cell-containing solution was spread on the LB agar that had been previously incubated at 37° C. for 30 minutes, and was incubated at 37° C. for 16 hours to obtain colonies.

### Preparation of E. coli-Containing Solution

For use as a template for the following PCR, an E. coli-containing solution was prepared and its concentration was re-adjusted after the measurement of the absorbance. That is, the plasmid extracted and purified from E. coli was not used as a template, but rather the dilution solution of E. coli per se was used as a template-containing nucleic acid solution.

Ten colonies were selected at random from the colonies obtained from the above-mentioned transformation, and were diluted in 100 μL of sterilized milli-Q water. After sufficient dilution, the absorbances at 601 nm and 280 nm were measured using the NanoDrop (registered trademark) ND-1000 Spectrophotometer (manufactured by NanoDrop). The absorbances at 601 nm and 280 nm were correlated. The concentration was adjusted through dilution so that the absorbance at 280 nm was set at 0.01. Thereafter, the obtained preparation solution was used as the template-containing nucleic acid solution.

### Preparation of Primers

The primer for discriminating the target base was used as the forward primer in which the target base-corresponding base and the mismatch base were respectively located at the second and the third base from the 3′ end. Specifically, the forward primers were divided into two major groups consisting of the asP-F-uXX (XX stands for a combination of two types of bases) series corresponding to the above-mentioned Mi-F-uXX series of the mutated pUC19 template, and the asP-F-dXX (XX stands for a combination of two types of bases) series corresponding to the above-mentioned Mi-F-dXX series thereof. The reverse primer used herein was the asP-R that was in common to every forward primer. The PCR amplification was performed by respective combination of the forward primer and the reverse primer. The details of these primers are shown in Table 2. In the table, among the underlined two bases in each sequence, the base on the 3′ side is the target base-corresponding base and the base on the 5′ side is the mismatch base.

TABLE 2

| | |
|---|---|
| primer | Sequence |

| | |
|---|---|
| A s p-F-uAA | 5′-TTAAGGGATTTTGGTCATGAGAAT-3′ |
| A s p-F-uAT | 5′-TTAAGGGATTTTGGTCATGAGATT-3′ |
| A s p-F-uAG | 5′-TTAAGGGATTTTGGTCATGAGAGT-3′ |
| A s p-F-uAC | 5′-TTAAGGGATTTTGGTCATGAGACT-3′ |
| A s p-F-uTA | 5′-TTAAGGGATTTTGGTGATGAGTAT-3′ |
| A s p-F-uTT | 5′-TTAAGGGATTTTGGTCATGAGTTT-3′ |
| A s p-F-uTG | 5′-TTAAGGGATTTTGGTCATGAGTGT-3′ |
| A s p-F-uTC | 5′-TTAAGGGATTTTGGTCATGAGTCT-3′ |
| A s p-F-uGA | 5′-TTAAGGGATTTTGGTCATGAGGAT-3′ |
| A s p-F-uGT | 5′-TTAAGGGATTTTGGTCATGAGGTT-3′ |
| A s p-F-uGG | 5′-TTAAGGGATTTTGGTCATGAGGGT-3′ |
| A s p-F-uGC | 5′-TTAAGGGATTTTGGTCATGAGGCT-3′ |
| A s p-F-uCA | 5′-TTAAGGGATTTTGGTCATGAGCAT-3′ |
| A s p-F-uCT | 5′-TTAAGGGATTTTGGTCATGAGCTT-3′ |
| A s p-F-uCG | 5′-TTAAGGGATTTTGGTCATGAGCGT-3′ |
| A s p-F-uCC | 5′-TTAAGGGATTTTGGTCATGAGCCT-3′ |
| A s p-F-dAA | 5′-TTAAGGGATTTTGGTCATGAAATT-3′ |
| A s p-F-dAT | 5′-TTAAGGGATTTTGGTCATGAATTT-3′ |
| A s p-F-dAG | 5′-TTAAGGGATTTTGGTCATGAAGTT-3′ |
| A s p-F-dAC | 5′-TTAAGGGATTTTGGTCATGAACTT-3′ |
| A s p-F-dTA | 5′-TTAAGGGATTTTGGTCATGATATT-3′ |
| A s p-F-dTT | 5′-TTAAGGGATTTTGGTCATGATTTT-3′ |
| A s p-F-dTG | 5′-TTAAGGGATTTTGGTCATGATGTT-3′ |
| A s p-F-dTC | 5′-TTAAGGGATTTTGGTCATGATCTT-3′ |
| A s p-F-dGA | 5′-TTAAGGGATTTTGGTCATGAGATT-3′ |
| A s p-F-dGT | 5′-TTAAGGGATTTTGGTCATGAGTTT-3′ |
| A s p-F-dGG | 5′-TTAAGGGATTTTGGTCATGAGGTT-3′ |
| A s p-F-dGC | 5′-TTAAGGGATTTTGGTCATGAGCTT-3′ |
| A s p-F-dCA | 5′-TTAAGGGATTTTGGTCATGACATT-3′ |
| A s p-F-dCT | 5′-TTAAGGGATTTTGGTCATGACTTT-3′ |
| A s p-F-dCG | 5′-TTAAGGGATTTTGGTCATGACGTT-3′ |
| A s p-F-dCC | 5′-TTAAGGGATTTTGGTCATGACCTT-3′ |
| A s p-R | 5′-CGAAATAGACAGATCGCTGAGATA-3′ |

### Evaluation of Specificity of Each Primer 1

The amount of the product decreases as the annealing temperature increases, and the specificity and the amplification efficiency can be evaluated using the degree of its decreasing rate. Therefore, PCR was performed under two conditions of the annealing temperature at 54° C. and at 58° C., and the ratio of the amplification product amount at 58° C. to the amplification product amount at 54° C. (p(58° C.)/p(54° C.)) was regarded as the fluctuation coefficient of the amplification product amount. On the basis of this fluctuation coefficient, each forward primer was evaluated for the specificity and the amplification efficiency for the target base in possible combinations between the base type of the mismatch base and the base type of the mismatch-corresponding base in the target nucleic acid corresponding thereto, when the target base was A.

Specifically, 3 μL of 2 mM dNTPs, 1.2 μL of 25 mM magnesium sulfate, 0.2 μL of 45 μM forward primer, 0.2 μL of 45 μM reverse primer, 5 μL of the nucleic acid sample solution, 0.6 μL of KOD Plus (1.0 U/μL), and an appropriate amount of sterile water were respectively added to 3 μL of 10×PCR buffer to prepare 30 μL of a reaction solution. Then, the reaction solution was subjected to the specificity evaluation PCR by treatment at 94° C. for 2 minutes, subsequent 35 thermal cycles at 94° C. for 30 seconds, 54° C. or 58° C. for 30 seconds, and 68° C. for 30 seconds, and an additional treatment at 68° C. for 2 minutes. The PCR apparatus was the Gradient Thermal Cycler PTC-200 manufactured by the former MJ Research, Inc. (currently, Bio-Rad Laboratories).

The nucleic acid sample solution and the forward primer used for each PCR are described in Table 3.

In Table 3, the term “(mutagenesis primer pair)” refers to a primer pair used in the mutagenesis PCR conducted for obtaining the nucleic acid sample solution for use as a template. In addition, the term “specificity evaluation primer pair” refers to a primer pair used in the specificity evaluation PCR.

TABLE 3

| Mismatch-corresponding | | Specificity | |
|---|---|---|---|
| base (mutagenesis | | evaluation primer | p(58° C.)/ |
| primer pair) | Target base | pair | p(54° C.) |
| A | A | asP-F-uAT, asP-R | 0.7 |
| Mi-F-uAA | | asP-F-uTT, asP-R* | 0.1 |
| Mi-R | | asP-F-uGT, asP-R | 0.7 |
| | | asP-F-uCT, asP-R | 0.6 |
| T | | asP-F-uAT, asP-R* | 0.3 |
| Mi-F-uTA | | asP-F-uTT, asP-R# | 0.2 |
| Mi-R | | asP-F-uGT, asP-R# | 0.3 |
| | | asP-F-uCT, asP-R | 0.8 |
| G | | asP-F-uAT, asP-R | 1.0 |
| Mi-F-uGA | | asP-F-uTT, asP-R# | 0.9 |
| Mi-R | | asP-F-uGT, asP-R | 1.1 |
| | | asP-F-uCT, asP-R* | 0.9 |
| C | | asP-F-uAT, asP-R | 0.6 |
| Mi-F-uCA | | asP-F-uTT, asP-R | 0.9 |
| Mi-R | | asP-F-uGT, asP-R* | 0.1 |
| | | asP-F-uCT, asP-R | 0.6 |

The amount of the nucleic acid amplification product in the reaction solution after PCR was measured by the electrophoretic method. Specifically, the measurement was performed using the DNA 1000 LabChip Kit (manufactured by Agilent) as the electrophoresis assay kit, and the 2100 Bioanalyzer (manufactured by Agilent) as the electrophoresis apparatus. The quantification result of the nucleic acid amplification product was automatically displayed on the apparatus.

Table 3 shows the ratio of the amplification product amount (ng/μL) at 58° C. to the amplification product amount (ng/μL) at 54° C. (p(58° C.)/p(54° C.)) which was calculated from the thus obtained quantification results of the nucleic acid amplification products.

The asterisked primer in the table represents a primer for use as a reference which is completely complementary to the non-mismatched target nucleic acid. That is, it can be determined that the target base discrimination ability is preserved or improved through mismatch introduction, if the p(58° C.)/p(54° C.) value is equal to or lower than the value of the primer (hashed primer in the table) which is completely complementary to the target nucleic acid.

From the results of Table 3, it is apparent that the base type of the target base can be discriminated more accurately by using a primer introduced with T or G as a mismatch base rather than by using a primer introduced with C as a mismatch base if the target base is A and the mismatch-corresponding base is T.

Similarly, it is apparent that the base type of the target base can be discriminated more accurately by using a primer introduced with T as a mismatch base rather than by using a primer introduced with A or G as a mismatch base if the target base is A and the mismatch-corresponding base is G.

### Evaluation of Specificity of Each Primer 2

Using the nucleic acid sample solution and the primers shown in Table 4, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the target base was T. The terms “(mutagenesis primer pair)” and “specificity evaluation primer pair” in Table 4 refer to the same meanings as those of Table 3.

From the results of Table 4, it is apparent that the base type of the target base can be discriminated more accurately by using a primer introduced with T or G as a mismatch base if the target base is T and the mismatch-corresponding base is T, by using a primer introduced with T as a mismatch base if the target base is T and the mismatch-corresponding base is G, and by using a primer introduced with A as a mismatch base if the target base is T and the mismatch-corresponding base is C, respectively, rather than by using a primer introduced with another base type.

TABLE 4

| Mismatch-corresponding | | Specificity | |
|---|---|---|---|
| base (mutagenesis | | evaluation primer | p(58° C.)/ |
| primer pair) | Target base | pair | p(54° C.) |
| A | T | asP-F-uAA, asP-R | 0.9 |
| Mi-F-uAT | | asP-F-uTA, asP-R* | 0.0 |
| Mi-R | | asP-F-uGA, asP-R | 0.9 |
| | | asP-F-uCA, asP-R | 0.3 |
| T | | asP-F-uAA, asP-R* | 0.8 |
| Mi-F-uTT | | asP-F-uTA, asP-R# | 0.1 |
| Mi-R | | asP-F-uGA, asP-R# | 0.6 |
| | | asP-F-uCA, asP-R | 1.4 |
| G | | asP-F-uAA, asP-R | 1.0 |
| Mi-F-uGT | | asP-F-uTA, asP-R# | 0.1 |
| Mi-R | | asP-F-uGA, asP-R | 0.5 |
| | | asP-F-uCA, asP-R* | 0.3 |
| C | | asP-F-uAA, asP-R# | 0.1 |
| Mi-F-uCT | | asP-F-uTA, asP-R | 0.8 |
| Mi-R | | asP-F-uGA, asP-R* | 0.3 |
| | | asP-F-uCA, asP-R | 0.4 |

### Evaluation of Specificity of Each Primer 3

Using the nucleic acid sample solution and the primers shown in Table 5, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the target base was G. The terms “(mutagenesis primer pair)” and “specificity evaluation primer pair” in Table 5 refer to the same meanings as those of Table 3.

From the results of Table 5, it is apparent that the base type of the target base can be discriminated more accurately by using a primer introduced with A or C as a mismatch base if the target base is G and the mismatch-corresponding base is A, by using a primer introduced with T or G as a mismatch base if the target base is G and the mismatch-corresponding base is T, by using a primer introduced with T as a mismatch base if the target base is G and the mismatch-corresponding base is G, and by using a primer introduced with A, T or C as a mismatch base if the target base is G and the mismatch-corresponding base is C, respectively, rather than by using a primer introduced with another base type.

TABLE 5

| Mismatch-corresponding | | Specificity | |
|---|---|---|---|
| base (mutagenesis | | evaluation primer | p(58° C.)/ |
| primer pair) | Target base | pair | p(54° C.) |
| A | G | asP-F-uAC, asP-R# | 0.0 |
| Mi-F-uAG | | asP-F-uTC, asP-R* | 0.0 |
| Mi-R | | asP-F-uGC, asP-R | 0.8 |
| | | asP-F-uCC, asP-R# | 0.0 |
| T | | asP-F-uAC, asP-R* | 0.6 |
| Mi-F-uTG | | asP-F-uTC, asP-R# | 0.2 |
| Mi-R | | asP-F-uGC, asP-R# | 0.6 |
| | | asP-F-uCC, asP-R | 0.8 |
| G | | asP-F-uAC, asP-R | 0.8 |
| Mi-F-uGG | | asP-F-uTC, asP-R# | 0.1 |
| Mi-R | | asP-F-uGC, asP-R | 0.7 |
| | | asP-F-uCC, asP-R* | 0.2 |
| C | | asP-F-uAC, asP-R# | 0.8 |
| Mi-F-uCG | | asP-F-uTC, asP-R# | 1.0 |
| Mi-R | | asP-F-uGC, asP-R* | 1.0 |
| | | asP-F-uCC, asP-R# | 0.8 |

### Evaluation of Specificity of Each Primer 4

Using the nucleic acid sample solution and the primers shown in Table 6, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the target base was C. The terms “(mutagenesis primer pair)” and “specificity evaluation primer pair” in Table 6 refer to the same meanings as those of Table 3.

From the results of Table 6, it is apparent that the base type of the target base can be discriminated more accurately by using a primer introduced with C as a mismatch base if the target base is C and the mismatch-corresponding base is A, by using a primer introduced with T or G as a mismatch base if the target base is C and the mismatch-corresponding base is T, by using a primer introduced with A as a mismatch base if the target base is C and the mismatch-corresponding base is G, and by using a primer introduced with C as a mismatch base if the target base is C and the mismatch-corresponding base is C, respectively, rather than by using a primer introduced with another base type.

TABLE 6

| Mismatch-corresponding | | Specificity | |
|---|---|---|---|
| base (mutagenesis | | evaluation primer | p(58° C.)/ |
| primer pair) | Target base | pair | p(54° C.) |
| A | C | asP-F-uAG, asP-R | 0.8 |
| Mi-F-uAC | | asP-F-uTG, asP-R* | 0.3 |
| Mi-R | | asP-F-uGG, asP-R | 0.9 |
| | | asP-F-uCG, asP-R# | 0.3 |
| T | | asP-F-uAG, asP-R* | 0.6 |
| Mi-F-uTC | | asP-F-uTG, asP-R# | 0.2 |
| Mi-R | | asP-F-uGG, asP-R# | 0.4 |
| | | asP-F-uCG, asP-R | 1.0 |
| G | | asP-F-uAG, asP-R# | 0.9 |
| Mi-F-uGC | | asP-F-uTG, asP-R | 1.1 |
| Mi-R | | asP-F-uGG, asP-R | 1.0 |
| | | asP-F-uCG, asP-R* | 0.9 |
| C | | asP-F-uAG, asP-R | 0.9 |
| Mi-F-uCC | | asP-F-uTG, asP-R | 1.1 |
| Mi-R | | asP-F-uGG, asP-R* | 0.6 |
| | | asP-F-uCG, asP-R# | 0.6 |

### Evaluation of Specificity of Each Primer 5

Using the nucleic acid sample solution and the primers shown in Table 7, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the anchor base was A. The terms “(mutagenesis primer pair)” and “specificity evaluation primer pair” in Table 7 refer to the same meanings as those of Table 3.

From the results of Table 7, it is apparent that the base type of the target base can be discriminated more accurately by using a primer introduced with C as a mismatch base if the anchor base is A and the mismatch-corresponding base is A, by using a primer introduced with G or C as a mismatch base if the anchor base is A and the mismatch-corresponding base is T, and by using a primer introduced with T as a mismatch base if the anchor base is A and the mismatch-corresponding base is C, respectively, rather than by using a primer introduced with another base type.

TABLE 7

| | Mismatch-corresponding | Specificity | |
|---|---|---|---|
| | base (mutagenesis | evaluation primer | p(58° C.)/ |
| Anchor base | primer pair) | pair | p(54° C.) |
| A | A | asP-F-dTA, asP-R | 0.8 |
| | Mi-F-dAA | asP-F-dTT, asP-R* | 0.2 |
| | Mi-R | asP-F-dTG, asP-R | 0.7 |
| | | asP-F-dTC, asP-R# | 0.0 |
| | T | asP-F-dTA, asP-R* | 0.8 |
| | Mi-F-dAT | asP-F-dTT, asP-R | 0.9 |
| | Mi-R | asP-F-dTG, asP-R# | 0.3 |
| | | asP-F-dTC, asP-R# | 0.0 |
| | G | asP-F-dTA, asP-R | 0.6 |
| | Mi-F-dAG | asP-F-dTT, asP-R | 0.2 |
| | Mi-R | asP-F-dTG, asP-R | 0.9 |
| | | asP-F-dTC, asP-R* | 0.0 |
| | C | asP-F-dTA, asP-R | 1.0 |
| | Mi-F-dAC | asP-F-dTT, asP-R# | 0.1 |
| | Mi-R | asP-F-dTG, asP-R* | 0.3 |
| | | asP-F-dTC, asP-R | 1.1 |

### Evaluation of Specificity of Each Primer 6

Using the nucleic acid sample solution and the primers shown in Table 8, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the anchor base was T. The terms “(mutagenesis primer pair)” and “specificity evaluation primer pair” in Table 8 refer to the same meanings as those of Table 3.

From the results of Table 8, it is apparent that the base type of the target base can be discriminated more accurately by using a primer introduced with C as a mismatch base if the anchor base is T and the mismatch-corresponding base is A, by using a primer introduced with G or C as a mismatch base if the anchor base is T and the mismatch-corresponding base is T, and by using a primer introduced with T as a mismatch base if the anchor base is T and the mismatch-corresponding base is C, respectively, rather than by using a primer introduced with another base type.

TABLE 8

| | Mismatch-corresponding | Specificity | |
|---|---|---|---|
| | base (mutagenesis | evaluation primer | p(58° C.)/ |
| Anchor base | primer pair) | pair | p(54° C.) |
| T | A | asP-F-dAA, asP-R | 0.8 |
| | Mi-F-dTA | asP-F-dAT, asP-R* | 0.1 |
| | Mi-R | asP-F-dAG, asP-R | 0.2 |
| | | asP-F-dAC, asP-R# | 0.0 |
| | T | asP-F-dAA, asP-R* | 0.4 |
| | Mi-F-dTT | asP-F-dAT, asP-R | 1.0 |
| | Mi-R | asP-F-dAG, asP-R# | 0.1 |
| | | asP-F-dAC, asP-R# | 0.0 |
| | G | asP-F-dAA, asP-R | 0.7 |
| | Mi-F-dTG | asP-F-dAT, asP-R | 0.2 |
| | Mi-R | asP-F-dAG, asP-R | 1.0 |
| | | asP-F-dAC, asP-R* | 0.0 |
| | C | asP-F-dAA, asP-R | 0.9 |
| | Mi-F-dTC | asP-F-dAT, asP-R# | 0.0 |
| | Mi-R | asP-F-dAG, asP-R* | 0.3 |
| | | asP-F-dAC, asP-R | 0.9 |

### Evaluation of Specificity of Each Primer 7

Using the nucleic acid sample solution and the primers shown in Table 9, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the anchor base was G. The terms “(mutagenesis primer pair)” and “specificity evaluation primer pair” in Table 9 refer to the same meanings as those of Table 3.

From the results of Table 9, it is apparent that the base type of the target base can be discriminated more accurately by using a primer introduced with G or C as a mismatch base if the anchor base is G and the mismatch-corresponding base is A, by using a primer introduced with G or C as a mismatch base if the anchor base is G and the mismatch-corresponding base is T, and by using a primer introduced with A or T as a mismatch base if the anchor base is G and the mismatch-corresponding base is C, respectively, rather than by using a primer introduced with another base type.

TABLE 9

| | Mismatch-corresponding | Specificity | |
|---|---|---|---|
| | base (mutagenesis | evaluation primer | p(58° C.)/ |
| Anchor base | primer pair) | pair | p(54° C.) |
| G | A | asP-F-dCA, asP-R | 0.8 |
| | Mi-F-dGA | asP-F-dCT, asP-R* | 0.1 |
| | Mi-R | asP-F-dCG, asP-R# | 0.1 |
| | | asP-F-dCC, asP-R# | 0.0 |
| | T | asP-F-dCA, asP-R* | 0.4 |
| | Mi-F-dGT | asP-F-dCT, asP-R | 0.8 |
| | Mi-R | asP-F-dCG, asP-R# | 0.2 |
| | | asP-F-dCC, asP-R# | 0.0 |
| | G | asP-F-dCA, asP-R | 0.7 |
| | Mi-F-dGG | asP-F-dCT, asP-R | 0.1 |
| | Mi-R | asP-F-dCG, asP-R | 0.9 |
| | | asP-F-dCC, asP-R* | 0.0 |
| | C | asP-F-dCA, asP-R# | 0.1 |
| | Mi-F-dGC | asP-F-dCT, asP-R# | 0.0 |
| | Mi-R | asP-F-dCG, asP-R* | 0.1 |
| | | asP-F-dCC, asP-R | 0.9 |

### Evaluation of Specificity of Each Primer 8

Using the nucleic acid sample solution and the primers shown in Table 10, each forward primer was evaluated for the specificity and the amplification efficiency for the target base, in the same manner as that of the evaluation of specificity of each primer 1, in possible combinations between the base type of the mismatch base and the base type of the mismatch-corresponding base in the target nucleic acid corresponding thereto, when the anchor base was C. The terms “(mutagenesis primer pair)” and “specificity evaluation primer pair” in Table 10 refer to the same meanings as those of Table 3.

From the results of Table 10, it is apparent that the base type of the target base can be discriminated more accurately by using a primer introduced with A, G, or C as a mismatch base if the anchor base is C and the mismatch-corresponding base is A, by using a primer introduced with C as a mismatch base if the anchor base is C and the mismatch-corresponding base is T, by using a primer introduced with A, T, or G as a mismatch base if the anchor base is C and the mismatch-corresponding base is G, and by using a primer introduced with A or T as a mismatch base if the anchor base is C and the mismatch-corresponding base is C, respectively, rather than by using a primer introduced with another base type.

TABLE 10

| | Mismatch-corresponding | Specificity | |
|---|---|---|---|
| | base (mutagenesis | evaluation primer | p(58° C.)/ |
| Anchor base | primer pair) | pair | p(54° C.) |
| C | A | asP-F-dGA, asP-R# | 1.0 |
| | Mi-F-dCA | asP-F-dGT, asP-R* | 1.0 |
| | Mi-R | asP-F-dGG, asP-R# | 1.0 |
| | | asP-F-dGC, asP-R# | 1.0 |
| | T | asP-F-dGA, asP-R* | 0.9 |
| | Mi-F-dCT | asP-F-dGT, asP-R | 1.0 |
| | Mi-R | asP-F-dGG, asP-R | 1.1 |
| | | asP-F-dGC, asP-R# | 0.8 |
| | G | asP-F-dGA, asP-R | 1.0 |
| | Mi-F-dCG | asP-F-dGT, asP-R | 1.0 |
| | Mi-R | asP-F-dGG, asP-R# | 0.7 |
| | | asP-F-dGC, asP-R* | 1.0 |
| | C | asP-F-dGA, asP-R# | 0.8 |
| | Mi-F-dCC | asP-F-dGT, asP-R# | 0.7 |
| | Mi-R | asP-F-dGG, asP-R* | 0.8 |
| | | asP-F-dGC, asP-R | 1.1 |

### SNP Detection Using Target Base-Specific Primer

SNP at the position 1173 (SNP ID: rs9934438 (1173 C>T)) in the human gene of vitamin K epoxide reductase complex (VKORC) subunit 1 (VKORC1), assuming A in the initiation codon (ATG) as the first position, was detected using the target base-specific primer of the present invention. The respective primers used herein were synthesized at SIGMA-ALDRICH Japan K.K., Life Science Division by order.

### <Confirmation of Genotype of Human Genome Specimen>

For use in the detection of VKORC1 (1173 C>T), the human genome specimens shown in Table 11 were obtained in an anonymous and unlinkable fashion from the Health Science Research Resources Bank provided by the Japan Health Sciences Foundation. The nucleotide sequences of these human genome specimens were read out with a sequencer and their genotypes were confirmed. The confirmed genotypes are shown in Table 11.

TABLE 11

| Human genome | |
|---|---|
| specimen | VKORC1 (1173 C > T) |
| PSCDA 0206 | T/T |
| PSCDA 0532 | T/T |
| PSCDA 0182 | C/T |
| PSCDA 0210 | C/T |
| PSCDA 0194 | C/T |
| PSCDA 0009 | T/T |
| PSCDA 0015 | T/T |
| PSCDA 0029 | T/T |

### <Primer Design>

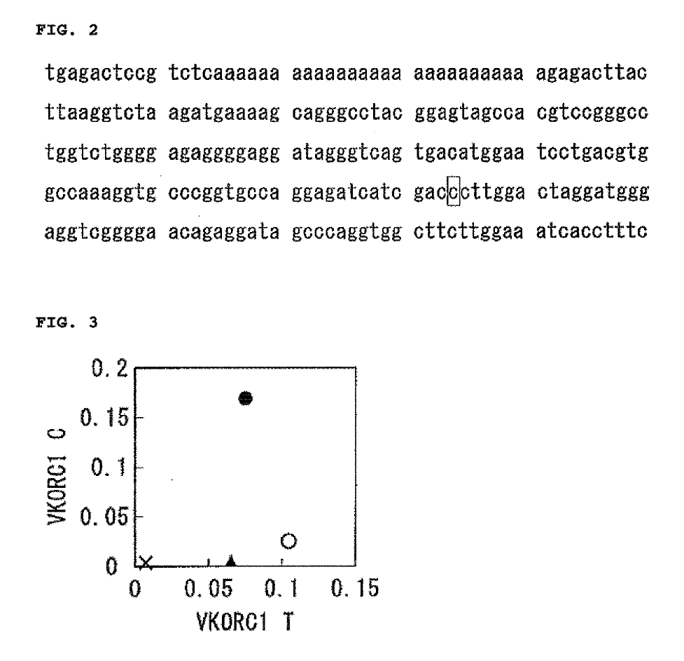

Primers were designed assuming that the target base was a base at the position 1173 of VKORC1 and the predicted base type was C (1173C) or T (1173T). The designed primers included a primer capable of specific detection of 1173C and a primer capable of specific detection of 1173T both serving as the target base-specific primers of the present invention, a primer capable of specific detection of 1173C not serving as the primer of the present invention, and a VKORC1-R primer as a reverse primer for performing PCR with any one of the above primers as a forward primer and the VKORC1 gene in the vicinity of VKORC1 (1173 C>T) as a template. FIG. 2 shows the nucleotide sequence of the VKORC1 gene in the vicinity of VKORC1 (1173 C>T). The base C in the square is the base at the position 1173 serving as the detection subject.

First, a primer for specific detection of 1173C was designed. As is apparent from the nucleotide sequence of the VKORC1 gene, the mismatch-corresponding base is C and the anchor base is T. Therefore, the above-mentioned rule (1m) for the primer according to the first aspect of the present invention instructs that the mismatch base is C, whereas the rule (2f) for the primer according to the second aspect of the present invention instructs that the mismatch base is T. Therefore, their base types are incompatible. In this Example, priority was given to the primer according to the second aspect of the present invention, and the mismatch base was set as T to design the VKORC1-C(T) primer.

Next, a primer for specific detection of 1173T was designed. In this case, the above-mentioned rule (1e) for the primer according to the first aspect of the present invention instructs that the mismatch base is A, whereas the rule (2f) for the primer according to the second aspect of the present invention instructs that the mismatch base is T. Therefore, their base types are incompatible. In this Example, priority was given to the primer according to the second aspect of the present invention, and the mismatch base was set as T to design the VKORC1-T(T) primer.

In addition, a primer capable of specific detection of 1173C not serving as the primer of the present invention, namely the VKORC1-C(A) primer, was designed to have the same nucleotide sequence as that of the VKORC1-C(T) primer except that the mismatch base was set as A.

Furthermore, the VKORC1-R primer which can function as a reverse primer with any one of these three primers as a forward primer, was designed.

The nucleotide sequences of the thus produced four types of primers are shown in Table 12.

TABLE 12

| | |
|---|---|
| primer | Sequence |

| | |
|---|---|
| VKORC 1-C(T) | 5′ - GACCTCCCATCCTAGTCCAATGG -3′ |
| VKORC 1-T(T) | 5′ - CGACCTCCCATCCTAGTCCAATAG -3′ |
| VKORC 1-C(A) | 5′ - GACCTCCCATCCTAGTCCAAAGG -3′ |
| VKORC 1-R | 5′ - ATGAAAAGCAGGGCCTACG-3′ |

### <Modification of Primer>

First, the VKORC1-C(T) primer and the VKORC1-T(T) primer having the nucleotide sequences of Table 12 were respectively modified with ligands to thereby produce a 5′ DIG-conjugated VKORC1-C(T) primer in which the 5′ end was conjugated with digoxigenin (DIG) and a 5′ DNP-conjugated VKORC1-T(T) primer in which the 5′ end was conjugated with dinitorophenol (DNP).

Next, the VKORC1-R primer having the nucleotide sequence of Table 12 was modified with a ligand to thereby produce a 5′ FITC-conjugated VKORC1-R primer in which the 5′ end was conjugated with fluorescein isothiocyanate (FITC).

### <Preparation of Receptor-Bindable Dispersible Fine Particles>

Dispersible fine particles conjugated with goat anti-DIG antibodies (manufactured by GeneTex, Inc.) which were specifically bindable to DIG (hereunder, anti-DIG dispersible fine particles), dispersible fine particles conjugated with goat anti-FITC antibodies (manufactured by BETHYL Laboratories, Inc.) which were specifically bindable to FITC (hereunder, anti-FITC dispersible fine particles), and dispersible fine particles conjugated with goat anti-DNP antibodies (manufactured by BETHYL Laboratories, Inc.) which were specifically bindable to DNP (hereunder, anti-DNP dispersible fine particles) were prepared.

Specifically, the respective receptor-bindable dispersible fine particles were prepared in the following manner. First, 1 mL of MES (manufactured by Wako) buffer solution (500 mM, pH6.1) was added with water until the volume reached 9 mL. The thus prepared solution was poured into a 50 mL tube, to which 1 mL of 10% CM-MP (300 nm Carboxyl latex particles, manufactured by Ceradyn) slurry was added. Then, 8 mL of 1 mg/mL each antibody phosphate buffer solution was added. The 50 mL tube was agitated for 5 seconds at a medium rotational strength of a vortex mixer (VORTEXGENIE2, manufactured by Scientific Industries). In addition, 1 mL of 1.152 g/L EDAC (MW=191.7, manufactured by Sigma) MES buffer solution (50 mM, pH 6.1) was added, and immediately thereafter the tube was agitated for 5 seconds at a medium rotational strength of the vortex mixer. Then, this 50 mL tube was set in a rotator (MTR-103, manufactured by AS ONE Corporation) and agitated at room temperature for 1 hour, followed by centrifugation at 4° C. at 15,000×g for 5 minutes. The centrifugation was stopped at a minimum deceleration condition so as to prevent the centrifugally separated CM-MP from floating up to the supernatant side. After the supernatant had been removed, 10 mL of MES buffer solution (50 mM, pH 6.1) was added, and the mixture was then subjected to ultrasonication at room temperature for 3 to 4 seconds with a strength of 30 amplitudes of a sonicator (VC-130, manufactured by Sonics) to thereby disperse the aggregation of CM-MP. The 50 mL tube was again centrifuged and added with 10 mL of MES buffer solution to disperse the aggregation of CM-MP in the same manner. Then, the resultant product was left standing at 4° C. for 16 hours, and was confirmed to contain no sediment by visual observation. The thus obtained CM-MP dispersed in the MES buffer solution was used as a 1% receptor-bindable dispersible fine particle slurry.

### <Detection of VKORC1 (1173 C>T) 1>

VKORC1 (1173 C>T) was detected in three types of human genome specimens (PSCDA0206, 0182, and 0009) using the 5′ DIG-conjugated VKORC1-C(T) primer, the 5′ DNP-conjugated VKORC1-T(T) primer, and the 5′ FITC-conjugated VKORC1-R primer.

First, 6 μL of 10× Buffer (manufactured by TOYOBO), 6 μL of dNTPs (2 mM), 2.4 μL of magnesium sulfate (25 mM), 10 μL of human genome specimen (20 ng/μL), 1.7 μL of the 5′ DIG-conjugated VKORC1-C(T) primer, 1.8 μL of the 5′ DNP-conjugated VKORC1-T(T) primer, 1.2 μL of the 5′ FITC-conjugated VKORC1-R primer, and 1.2 μL of KOD plus (manufactured by TOYOBO) were added to 29.7 μL of milli-Q water to prepare a reaction solution. The reaction solution was subjected to PCR under the reaction condition consisting of a denaturation step at 94° C. for 5 minutes, a subsequent amplification step of 35 cycles at 94° C. for 30 seconds, 64° C. for 30 seconds, and 68° C. for 30 seconds, and an extension step at 68° C. for 2 minutes. An equivalent volume of milli-Q water instead of the human genome specimen was used as a negative control.

Then, the presence or absence of PCR amplification was examined through immune nephelometry using the receptor-bindable dispersible fine particles. First, the presence or absence of PCR amplification with the 5′ DIG-conjugated VKORC1-C(T) primer was examined in each PCR-treated reaction solution. Respectively, 1 μL of 1% anti-DIG dispersible fine particle slurry and 1 μL of 1% anti-FITC dispersible fine particle slurry were added to 2 μL of each PCR-treated reaction solution. The mixture was agitated with a vortex mixer, and left at room temperature for 5 minutes. The absorbance at 800 nm (A800) was respectively measured before the addition of the dispersible fine particles into the PCR-treated reaction solution and after the formation of aggregation using a spectrophotometer (SPECTRA max PLUS384, manufactured by Molecular Device), to measure the change in the A800 value of the PCR-treated reaction solution due to the addition of the dispersible fine particles.

Next, in order to examine the presence or absence of PCR amplification with the 5′ DNP-conjugated VKORC1-T(T) primer in each PCR-treated reaction solution, 1 μL of 1% anti-DNP dispersible fine particle slurry and 1 μL of 1% anti-FITC dispersible fine particle slurry were respectively added to 2 μL of each PCR-treated reaction solution, followed by measurement of change in the A800 value of the PCR-treated reaction solution due to the addition of the dispersible fine particles in the same manner.

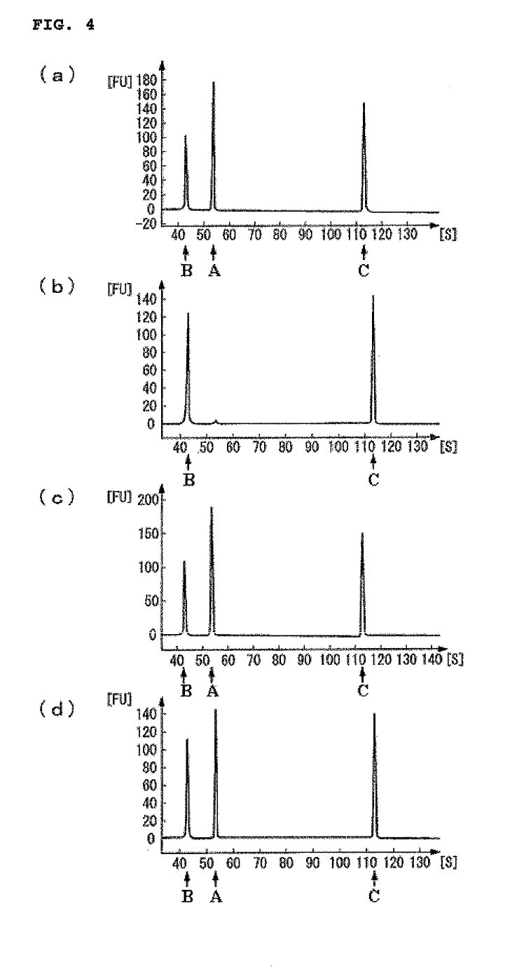

FIG. 3 is a cluster diagram showing the detection results of VKORC1 (1173 C>T). The y-axis shows the change in the A800 value of respective PCR-treated reaction solutions after the addition of the anti-DIG dispersible fine particles and the anti-FITC dispersible fine particles, and the x-axis shows the change in the A800 value of respective PCR-treated reaction solutions after the addition of the anti-DNP dispersible fine particles and the anti-FITC dispersible fine particles. The solid circle represents the result of PSCDA0182, the open circle represents the result of PSCDA0206, the solid triangle represents the result of PSCDA0009, and the mark x represents the result of the negative control.

The A800 value was increased by the addition of the anti-DNP dispersible fine particles in all three types of human genome specimens. On the other hand, the A800 value was increased by the addition of the anti-DIG dispersible fine particles in PSCDA0182, while no great change was observed in the other two types of human genome specimens. Furthermore, almost no change in the A800 value was observed in both cases of the negative control. That is, it was found that the PCR amplification with the 5′ DNP-conjugated VKORC1-T (T) primer was successful in all three types of human genome specimens, whereas the PCR amplification with the 5′ DIG-conjugated VKORC1-C(T) primer was only successful in PSCDA0182. That is to say, the results indicated that PSCDA0182 was VKORC1 (1173C/T), and PSCDA0206 and 0009 were VKORC1 (1173T/T). The results agreed with the results of polymorphism discrimination through sequence analysis, and it is apparent that VKORC1 (1173 C>T) can be detected by using the VKORC1-C(T) primer and the VKORC1-T(T) primer serving as the target base-specific primers of the present invention.

### <Detection of VKORC1 (1173 C>T) 2>

PCR was respectively performed using the VKORC1-C(T) primer or the VKORC1-C(A) primer having the nucleotide sequences of Table 12 as a forward primer, the VKORC1-R primer as a reverse primer, and two types of human genome specimen (PSCDA0210 and 0532) as a template.

Specifically, first, 6 μL of 5× Buffer (manufactured by TOYOBO), 5 μL of dNTPs (2 mM), 2 μL of magnesium sulfate (25 mM), 1 μL of human genome specimen (20 ng/μL), 2.5 μL of the forward primer, 2.5 μL of the reverse primer, and 1 μL of KOD plus (manufactured by TOYOBO) were added to 31 μL of milli-Q water to prepare a reaction solution. The reaction solution was subjected to PCR under the same reaction condition as that of <Detection of VKORC1 (1173 C>T) 1>. The resultant PCR products were separated by agarose electrophoresis, followed by the observation of their band patterns using the Agilent 2100 BIOANALYZER (DNA1000kit, manufactured by Agilent Technology).

The obtained band patterns are shown in FIG. 4. (a) shows the band pattern of the PCR product resulting from the VKORC1-C(T) primer and PSCDA0210, (b) shows the band pattern of the PCR product resulting from the VKORC1-C(T) primer and PSCDA0532, (c) shows the band pattern of the PCR product resulting from the VKORC1-C(A) primer and PSCDA0210, and (d) shows the band pattern of the PCR product resulting from the VKORC1-C(A) primer and PSCDA0532. The arrow A shows a band of the PCR product, and the arrows B and C show bands of markers. Bands of PCR products were detected in (a), (c), and (d).

The results of the sequence analysis indicated that PSCDA0210 was VKORC1 (1173C/T) and PSCDA0532 was VKORC1 (1173T/T). In fact, the PCR product was detected in PSCDA0210 and no PCR product was detected in PSCDA0532 with the VKORC1-C(T) primer. On the other hand, the PCR products were detected in both PSCDA0210 and PSCDA0532 with the VKORC1-C(A) primer.

That is, nonspecific PCR amplification had occurred due to misextension from the VKORC1-C(A) primer.

From these results, it is apparent that the target base-specific primer of the present invention, in which the base type of the mismatch base of the primer is selected from the above-mentioned (1a) to (1m) and/or (2a) to (2m), greatly excels in the specificity.

### INDUSTRIAL APPLICABILITY

The target base-specific primer of the present invention is capable of discriminating the base type of the target base clearly with much less possibility of false positive. Therefore application is possible in the field of genetic analysis, such as SNP analysis, where a single-base difference has to be discriminated with high accuracy, in medical institutions of genetic analysis and the like.

1. A target base-specific primer for use in discrimination of a base type of a target base in a nucleic acid having a target nucleotide sequence including the target base in a nucleic acid sample, in the case where a plurality of base types can be enumerated for the target base, wherein
the primer has a target base-corresponding base which is a base complementary to a base predicted as the target base, and
the base type of a mismatch base is a base selected from the group consisting of the following (1a) to (1m) when the mismatch base refers to a base adjacent to the 5′ side of the target base-corresponding base, and a mismatch-corresponding base refers to a base adjacent to the 3′ side of the target base in the target nucleotide sequence:
(1a) if the base predicted as the target base is A and the mismatch-corresponding base is T, the mismatch base is either T or G;
(1b) if the base predicted as the target base is A and the mismatch-corresponding base is C, the mismatch base is T;
(1c) if the base predicted as the target base is T and the mismatch-corresponding base is T, the mismatch base is either T or G;
(1d) if the base predicted as the target base is T and the mismatch-corresponding base is G, the mismatch base is T;
(1e) if the base predicted as the target base is T and the mismatch-corresponding base is C, the mismatch base is A;
(1f) if the base predicted as the target base is G and the mismatch-corresponding base is A, the mismatch base is either A or C;
(1g) if the base predicted as the target base is G and the mismatch-corresponding base is T, the mismatch base is either T or G;
(1h) if the base predicted as the target base is G and the mismatch-corresponding base is G, the mismatch base is T;
(1i) if the base predicted as the target base is G and the mismatch-corresponding base is C, the mismatch base is any one of A, T, and C;
(1j) if the base predicted as the target base is C and the mismatch-corresponding base is A, the mismatch base is C;
(1k) if the base predicted as the target base is C and the mismatch-corresponding base is T, the mismatch base is either T or a;
(1l) if the base predicted as the target base is C and the mismatch-corresponding base is G, the mismatch base is A; and
(1m) if the base predicted as the target base is C and the mismatch-corresponding base is C, the mismatch base is C.

2. A target base-specific primer for use in discrimination of a base type of a target base in a nucleic acid having a target nucleotide sequence including the target base in a nucleic acid sample, in the case where a plurality of base types can be enumerated for the target base, wherein
the primer has a target base-corresponding base which is a base complementary to a base predicted as the target base, and
the base type of a mismatch base is a base selected from the group consisting of the following (2a) to (2m) when the mismatch base refers to a base adjacent to the 5′ side of the target base-corresponding base, an anchor base refers to a base adjacent to the 3′ side of the mismatch-corresponding base, and a mismatch-corresponding base refers to a base adjacent to the 3′ side of the target base in the target nucleotide sequence:
(2a) if the anchor base is A and the mismatch-corresponding base is A, the mismatch base is C;
(2b) if the anchor base is A and the mismatch-corresponding base is T, the mismatch base is either G or C;
(2c) if the anchor base is A and the mismatch-corresponding base is C, the mismatch base is T;
(2d) if the anchor base is T and the mismatch-corresponding base is A, the mismatch base is C;
(2e) if the anchor base is T and the mismatch-corresponding base is T, the mismatch base is either G or C;
(2f) if the anchor base is T and the mismatch-corresponding base is C, the mismatch base is T;
(2g) if the anchor base is G and the mismatch-corresponding base is A, the mismatch base is either G or C;
(2h) if the anchor base is G and the mismatch-corresponding base is T, the mismatch base is either G or C;
(2i) if the anchor base is G and the mismatch-corresponding base is C, the mismatch base is either A or T;
(2j) if the anchor base is C and the mismatch-corresponding base is A, the mismatch base is any one of A, G, and C;
(2k) if the anchor base is C and the mismatch-corresponding base is T, the mismatch base is C;
(2l) if the anchor base is C and the mismatch-corresponding base is G, the mismatch base is any one of A, T, and a; and
(2m) if the anchor base is C and the mismatch-corresponding base is C, the mismatch base is either A or T.

3. The target base-specific primer according to claim 2, wherein the base type of said mismatch base is a base selected from the group consisting of the following (1a) to (1m):
(1a) if the base predicted as the target base is A and the mismatch-corresponding base is T, the mismatch base is either T or G;
(1b) if the base predicted as the target base is A and the mismatch-corresponding base is G, the mismatch base is T;
(1c) if the base predicted as the target base is T and the mismatch-corresponding base is T, the mismatch base is either T or C;
(1d) if the base predicted as the target base is T and the mismatch-corresponding base is G, the mismatch base is T;
(1e) if the base predicted as the target base is T and the mismatch-corresponding base is C, the mismatch base is A;
(1f) if the base predicted as the target base is G and the mismatch-corresponding base is A, the mismatch base is either A or C;
(1g) if the base predicted as the target base is G and the mismatch-corresponding base is T, the mismatch base is either T or G;
(1h) if the base predicted as the target base is G and the mismatch-corresponding base is G, the mismatch base is T;
(1i) if the base predicted as the target base is G and the mismatch-corresponding base is C, the mismatch base is any one of A, T, and C;
(1j) if the base predicted as the target base is C and the mismatch-corresponding base is A, the mismatch base is C;
(1k) if the base predicted as the target base is C and the mismatch-corresponding base is T, the mismatch base is either T or G;
(1l) if the base predicted as the target base is C and the mismatch-corresponding base is G, the mismatch base is A; and
(1m) if the base predicted as the target base is C and the mismatch-corresponding base is C, the mismatch base is C.

4. The target base-specific primer according to claim 3, wherein
said mismatch base consists of one base, and
the base type of the mismatch base is the base selected from the group consisting of (2a) to (2m) if the base type of the mismatch base is different between the base selected from the group consisting of (1a) to (1m) and the base selected from the group consisting of (2a) to (2m).

5. The target base-specific primer according to claim 1, wherein said mismatch base(s) and said mismatch-corresponding base(s) consist of one or two base(s).

6. The target base-specific primer according to claim 1, wherein said mismatch base is located within four bases from the 3′ end.

7. A target base discrimination method which discriminates the base type of a target base in a nucleic acid having a target nucleotide sequence including the target base in a nucleic acid sample, in the case where a plurality of base types can be enumerated for the target base, comprising the steps of:
(I) hybridizing the nucleic acid in the nucleic acid sample and the target base-specific primer according to claim 1, and performing a nucleic acid extension reaction; and
(II) discriminating the base type of the target base included in the nucleic acid in the nucleic acid sample on the basis of the nucleic acid extension efficiency of said step (I).

8. The target base discrimination method according to claim 7, wherein
said step (II) is
a step (II-1) of determining that the base type of the target base included in the nucleic acid in the nucleic acid sample is the predicted base if the nucleic acid extension efficiency of said step (I) is high, and that the base type of the target base is different from the predicted base if the nucleic acid extension efficiency of said step (I) is low.

9. The target base discrimination method according to claim 7, further comprising:
after said step (1) and before said step (II),
a (II) of hybridizing the nucleic acid having a target nucleotide sequence in which the base type of the target base is different from the predicted base and the target base-specific primer, and performing a nucleic acid extension reaction, and
said step (II) is
a step (II-2) of determining that the base type of the target base included in the nucleic acid in the nucleic acid sample is the predicted base if the amount of the extension product resulting from said step (I) is greater than the amount of the extension product resulting from said step (III), and that the base type of the target base is different from the predicted base if the amount of the extension product resulting from said step (I) is not greater than the amount of the extension product resulting from said step (III).

10. The target base discrimination method according to claim 9, wherein
said step (II-2) is
a step (II-3) of determining that the base type of the target base included in the nucleic acid in the nucleic acid sample is the predicted base if the amount of the extension product resulting from said step (I) is three or more times greater than the amount of the extension product resulting from said step (III), and that the base type of the target base is different from the predicted base if the amount of the extension product resulting from said step (I) is less than three times the amount of the extension product resulting from said step (III).

11. The target base discrimination method according to claim 7, wherein said nucleic acid extension reaction is a primer extension reaction.

12. The target base discrimination method according to claim 1, wherein said primer extension reaction is a nucleic acid amplification reaction selected from the group consisting of a PCR (Polymerase Chain Reaction) method, a LAMP (Loop-Mediated Isothermal Amplification) method, a SMAP (SMart Amplification Process) method, an RCR (Recombinatorial Chain Reaction) method, a TMA (Transcripition mediated amplification) method, a PALSAR (Probe alternation link self-assembly reaction) method, a NASBA (nucleic acid sequence based amplification) method, and an ICAN (Isothermal and chimeric primer-initiated amplification of nucleic acids) method.

13. The target base discrimination method according to claim 7, wherein said nucleic acid extension efficiency is examined on the basis of the amount of the amplification product yielded from said nucleic acid amplification reaction, in said step (II).

14. The target base discrimination method according to claim 7, wherein said nucleic acid extension efficiency is examined on the basis of the molecular weight of the amplification product yielded from said nucleic acid amplification reaction, in said step (II).


SEQUENCE LISTING (70 sequences)

SEQ ID NO: 1 - DNA, 35 bp, Artificial Sequence
  Mi-F-uAA primer
ttaagggattttggtcatgagaatatcaaaaagga

SEQ ID NO: 2 - DNA, 35 bp, Artificial Sequence
  Mi-F-uAT primer
ttaagggattttggtcatgagattatcaaaaagga

SEQ ID NO: 3 - DNA, 35 bp, Artificial Sequence
  Mi-F-uAG primer
ttaagggattttggtcatgagagtatcaaaaagga

SEQ ID NO: 4 - DNA, 35 bp, Artificial Sequence
  Mi-F-uAC primer
ttaagggattttggtcatgagactatcaaaaagga

SEQ ID NO: 5 - DNA, 35 bp, Artificial Sequence
  Mi-F-uTA primer
ttaagggattttggtcatgagtatatcaaaaagga

SEQ ID NO: 6 - DNA, 35 bp, Artificial Sequence
  Mi-F-uTT primer
ttaagggattttggtcatgagtttatcaaaaagga

SEQ ID NO: 7 - DNA, 35 bp, Artificial Sequence
  Mi-F-uTG primer
ttaagggattttggtcatgagtgtatcaaaaagga

SEQ ID NO: 8 - DNA, 35 bp, Artificial Sequence
  Mi-F-uTC primer
ttaagggattttggtcatgagtctatcaaaaagga

SEQ ID NO: 9 - DNA, 35 bp, Artificial Sequence
  Mi-F-uGA primer
ttaagggattttggtcatgaggatatcaaaaagga

SEQ ID NO: 10 - DNA, 35 bp, Artificial Sequence
  Mi-F-uGT primer
ttaagggattttggtcatgaggttatcaaaaagga

SEQ ID NO: 11 - DNA, 35 bp, Artificial Sequence
  Mi-F-uGG primer
ttaagggattttggtcatgagggtatcaaaaagga

SEQ ID NO: 12 - DNA, 35 bp, Artificial Sequence
  Mi-F-uGC primer
ttaagggattttggtcatgaggctatcaaaaagga

SEQ ID NO: 13 - DNA, 35 bp, Artificial Sequence
  Mi-F-uCA primer
ttaagggattttggtcatgagcatatcaaaaagga

SEQ ID NO: 14 - DNA, 35 bp, Artificial Sequence
  Mi-F-uCT primer
ttaagggattttggtcatgagcttatcaaaaagga

SEQ ID NO: 15 - DNA, 35 bp, Artificial Sequence
  Mi-F-uCG primer
ttaagggattttggtcatgagcgtatcaaaaagga

SEQ ID NO: 16 - DNA, 35 bp, Artificial Sequence
  Mi-F-uCC primer
ttaagggattttggtcatgagcctatcaaaaagga

SEQ ID NO: 17 - DNA, 35 bp, Artificial Sequence
  Mi-F-dAA primer
ttaagggattttggtcatgaaattatcaaaaagga

SEQ ID NO: 18 - DNA, 35 bp, Artificial Sequence
  Mi-F-dAT primer
ttaagggattttggtcatgaatttatcaaaaagga

SEQ ID NO: 19 - DNA, 35 bp, Artificial Sequence
  Mi-F-dAG primer
ttaagggattttggtcatgaagttatcaaaaagga

SEQ ID NO: 20 - DNA, 35 bp, Artificial Sequence
  Mi-F-dAC pr
ttaagggattttggtcatgaacttatcaaaaagga

SEQ ID NO: 21 - DNA, 35 bp, Artificial Sequence
  Mi-F-dTA primer
ttaagggattttggtcatgatattatcaaaaagga

SEQ ID NO: 22 - DNA, 35 bp, Artificial Sequence
  Mi-F-dTT primer
ttaagggattttggtcatgattttatcaaaaagga

SEQ ID NO: 23 - DNA, 35 bp, Artificial Sequence
  Mi-F-dTG primer
ttaagggattttggtcatgatgttatcaaaaagga

SEQ ID NO: 24 - DNA, 35 bp, Artificial Sequence
  Mi-F-dTC primer
ttaagggattttggtcatgatcttatcaaaaagga

SEQ ID NO: 25 - DNA, 35 bp, Artificial Sequence; identical to SEQ ID NO: 2
  Mi-F-dGA primer

SEQ ID NO: 26 - DNA, 35 bp, Artificial Sequence; identical to SEQ ID NO: 6
  Mi-F-dGT primer

SEQ ID NO: 27 - DNA, 35 bp, Artificial Sequence; identical to SEQ ID NO: 10
  Mi-F-dGG primer

SEQ ID NO: 28 - DNA, 35 bp, Artificial Sequence; identical to SEQ ID NO: 14
  Mi-F-dGC primer

SEQ ID NO: 29 - DNA, 35 bp, Artificial Sequence
  Mi-F-dCA primer
ttaagggattttggtcatgacattatcaaaaagga

SEQ ID NO: 30 - DNA, 35 bp, Artificial Sequence
  Mi-F-dCT primer
ttaagggattttggtcatgactttatcaaaaagga

SEQ ID NO: 31 - DNA, 35 bp, Artificial Sequence
  Mi-F-dCG primer
ttaagggattttggtcatgacgttatcaaaaagga

SEQ ID NO: 32 - DNA, 35 bp, Artificial Sequence
  Mi-F-dCC primer
ttaagggattttggtcatgaccttatcaaaaagga

SEQ ID NO: 33 - DNA, 33 bp, Artificial Sequence
  Mi-R primer
acgaaaactcacgttaagggattttggtcatga

SEQ ID NO: 34 - DNA, 24 bp, Artificial Sequence
  Asp-F-uAA primer
ttaagggattttggtcatgagaat

SEQ ID NO: 35 - DNA, 24 bp, Artificial Sequence
  Asp-F-uAT primer
ttaagggattttggtcatgagatt

SEQ ID NO: 36 - DNA, 24 bp, Artificial Sequence
  Asp-F-uAG primer
ttaagggattttggtcatgagagt

SEQ ID NO: 37 - DNA, 24 bp, Artificial Sequence
  Asp-F-uAC primer
ttaagggattttggtcatgagact

SEQ ID NO: 38 - DNA, 24 bp, Artificial Sequence
  Asp-F-uTA primer
ttaagggattttggtcatgagtat

SEQ ID NO: 39 - DNA, 24 bp, Artificial Sequence
  Asp-F-uTT primer
ttaagggattttggtcatgagttt

SEQ ID NO: 40 - DNA, 24 bp, Artificial Sequence
  Asp-F-uTG primer
ttaagggattttggtcatgagtgt

SEQ ID NO: 41 - DNA, 24 bp, Artificial Sequence
  Asp-F-uTC primer
ttaagggattttggtcatgagtct

SEQ ID NO: 42 - DNA, 24 bp, Artificial Sequence
  Asp-F-uGA primer
ttaagggattttggtcatgaggat

SEQ ID NO: 43 - DNA, 24 bp, Artificial Sequence
  Asp-F-uGT primer
ttaagggattttggtcatgaggtt

SEQ ID NO: 44 - DNA, 24 bp, Artificial Sequence
  Asp-F-uGG primer
ttaagggattttggtcatgagggt

SEQ ID NO: 45 - DNA, 24 bp, Artificial Sequence
  Asp-F-uGC primer
ttaagggattttggtcatgaggct

SEQ ID NO: 46 - DNA, 24 bp, Artificial Sequence
  Asp-F-uCA primer
ttaagggattttggtcatgagcat

SEQ ID NO: 47 - DNA, 24 bp, Artificial Sequence
  Asp-F-uCT primer
ttaagggattttggtcatgagctt

SEQ ID NO: 48 - DNA, 24 bp, Artificial Sequence
  Asp-F-uCG primer
ttaagggattttggtcatgagcgt

SEQ ID NO: 49 - DNA, 24 bp, Artificial Sequence
  Asp-F-uCC primer
ttaagggattttggtcatgagcct

SEQ ID NO: 50 - DNA, 24 bp, Artificial Sequence
  Asp-F-dAA primer
ttaagggattttggtcatgaaatt

SEQ ID NO: 51 - DNA, 24 bp, Artificial Sequence
  Asp-F-dAT primer
ttaagggattttggtcatgaattt

SEQ ID NO: 52 - DNA, 24 bp, Artificial Sequence
  Asp-F-dAG primer
ttaagggattttggtcatgaagtt

SEQ ID NO: 53 - DNA, 24 bp, Artificial Sequence
  Asp-F-dAC primer
ttaagggattttggtcatgaactt

SEQ ID NO: 54 - DNA, 24 bp, Artificial Sequence
  Asp-F-dTA primer
ttaagggattttggtcatgatatt

SEQ ID NO: 55 - DNA, 24 bp, Artificial Sequence
  Asp-F-dTT primer
ttaagggattttggtcatgatttt

SEQ ID NO: 56 - DNA, 24 bp, Artificial Sequence
  Asp-F-dTG primer
ttaagggattttggtcatgatgtt

SEQ ID NO: 57 - DNA, 24 bp, Artificial Sequence
  Asp-F-dTC primer
ttaagggattttggtcatgatctt

SEQ ID NO: 58 - DNA, 24 bp, Artificial Sequence
  Asp-F-dGA primer
ttaagggattttggtcatgagatt

SEQ ID NO: 59 - DNA, 24 bp, Artificial Sequence
  Asp-F-dGT primer
ttaagggattttggtcatgagttt

SEQ ID NO: 60 - DNA, 24 bp, Artificial Sequence
  Asp-F-dGG primer
ttaagggattttggtcatgaggtt

SEQ ID NO: 61 - DNA, 24 bp, Artificial Sequence
  Asp-F-dGC primer
ttaagggattttggtcatgagctt

SEQ ID NO: 62 - DNA, 24 bp, Artificial Sequence
  Asp-F-dCA primer
ttaagggattttggtcatgacatt

SEQ ID NO: 63 - DNA, 24 bp, Artificial Sequence
  Asp-F-dCT primer
ttaagggattttggtcatgacttt

SEQ ID NO: 64 - DNA, 24 bp, Artificial Sequence
  Asp-F-dCG primer
ttaagggattttggtcatgacgtt

SEQ ID NO: 65 - DNA, 24 bp, Artificial Sequence
  Asp-F-dCC primer
ttaagggattttggtcatgacctt

SEQ ID NO: 66 - DNA, 24 bp, Artificial Sequence
  Asp-R primer
cgaaatagacagatcgctgagata

SEQ ID NO: 67 - DNA, 23 bp, Artificial Sequence
  VKORC1-C(T) primer
gacctcccatcctagtccaatgg

SEQ ID NO: 68 - DNA, 24 bp, Artificial Sequence
  VKORC1-T(T) primer
cgacctcccatcctagtccaatag

SEQ ID NO: 69 - DNA, 23 bp, Artificial Sequence
  VKORC1-C(A) primer
gacctcccatcctagtccaaagg

SEQ ID NO: 70 - DNA, 19 bp, Artificial Sequence
  VKORC1-R primer
atgaaaagcagggcctacg